(19) United States
(12) Patent Application Publication
(10) Pub. No.: US 2009/0031436 A1
(43) Pub. Date: Jan. 29, 2009

(54) Compositions and Methods Relating to Cornelia De Lange Syndrome

(75) Inventors: Arthur Lander, Laguna Beach, CA (US); Anne L. Calof, Laguna Beach, CA (US)

Correspondence Address: FISH & ASSOCIATES, PC;ROBERT D. FISH, 2603 Main Street, Suite 1050, Irvine, CA, 92614-6232, US

(73) Assignee: THE REGENTS OF THE UNIVERSITY OF CALIFORNIA, Oakland, CA (US)

(21) Appl. No.: 12/088,642

(22) PCT Filed: Sep. 28, 2006
(86) PCT No.: PCT/US06/38178

§ 371 Date: Sep. 18, 2008

Related U.S. Application Data
(60) Provisional application No. 60/722,695, filed on Sep. 29, 2005.

(51) Int. Cl. C12Q 1/68; A01K 67/027

(52) U.S. Cl. 800/3; 800/9; 435/6

(57) ABSTRACT

Animal models, kits, and methods for diagnosis and treatment of Cornelia de Lange Syndrome are disclosed. In especially preferred aspects, altered gene expression of selected genes is correlated with a NIPBL+/− genotype to thereby identify surrogate markers, which may then be used to diagnose Cornelia de Lange Syndrome.

This application claims priority to our copending U.S. provisional patent application with the Ser. No. 60/722,695, filed Sep. 29, 2005, and which is incorporated by reference herein.

### FIELD OF THE INVENTION

The field of the invention is compositions and methods for treatment and diagnosis of Cornelia de Lange Syndrome.

### BACKGROUND OF THE INVENTION

Diagnosis of Cornelia de Lange Syndrome is typically performed postnatally and often relies on clinically apparent signs, including abnormal upper limbs and facial features, cardiac septal defects, pyloric stenosis, growth and cognitive retardation, and hearing loss. However, accurate diagnosis is complicated by the wide range of clinical signs, and in most cases, mild forms are frequently not identified. Thus, prevalence estimates vary between about 1:10,000 to about 1:40,000. Further details on clinical, genetic, and biochemical aspects of CdLS can be found in Online Mendelian Inheritance in Man (McKusick et al., Johns Hopkins, available via NCBI) using accession number #122470, which is incorporated by reference herein.

More recently, Cornelia de Lange Syndrome (CdLS) was found to be associated with changes in the NIPBL gene, which at least at first glance appeared to provide an objective and quantitative diagnostic target to diagnose a patient as affected by CdLS. However, CdLS has proven to be associated with numerous, diverse, and widespread mutations within the NIPBL gene (see e.g., Am. J. Hum. Genet. 75:610-623, 2004), and therefore necessitates sequencing of essentially the entire NIPBL gene to confirm or establish diagnosis. Unfortunately, the human NIPBL gene is a large gene (>130 kb, at least 47 exons, transcript of up to 9.8 kb, extensive 5′-UTR) and sequencing the entire gene is therefore often not performed. Alternatively, the NIPBL gene can be screened for selected mutations, but such an approach is typically inaccurate due to the large number (hundreds) of currently known mutations, and the high variability of pathologies associated with such mutations. For example, it has been demonstrated that the same mutation occurring in different patients was associated with substantially different degrees in pathology. On the other hand, it was also shown that patients with the same pathology will have in many cases different mutations in different loci. Still further, some pathologies common in CdLS patients (e.g., autistic behavior) show no correlation with the type of mutations that have been found in the NIPBL gene.

Underlying some of these problems is the fact CdLS is a haploinsufficiency syndrome; that is, mutation in one of the two alleles of the NIPBL gene is sufficient to produce the syndrome. Since only individuals with the mildest forms of CdLS usually reproduce, what is observed clinically is that the vast majority of CdLS-causing NIPBL mutations are newly arisen in the affected individual or the gametes of his or her parents. It is thus not effective to attempt to make a diagnosis of CdLS by screening for known mutations.

To complicate matters even further, the role of the NIPBL gene product as an apparently non-sequence-specific chromosomal protein, which regulates DNA structure to facilitate transcription of many genes, points to no particular and unambiguously identifiable relationship of NIPBL to individual physiological functions. Worse yet, the NIPBL gene appears to be ubiquitously expressed, albeit at different levels in all human tissues, so that alterations in NIPBL function could have a significant impact on potentially every tissue in the body. Moreover, the fact that CdLS appears to be caused by only partial loss of function of NIPBL (e.g. mutation of only one allele) suggests that information obtained from the study of complete loss of NIPBL function in lower organisms may not be particularly relevant to CdLS.

Non-genetic tests for CdLS have been proposed based on the observation that serum levels of PAPP-A (Pregnancy Associated Plasma Protein-A) are sometimes reduced in pregnancies where the mother caries a child later diagnosed with CdLS (see Prenat Diagn. 1983 July; 3(3):225-32). However, subsequent clinical studies have shown that changes in PAPP-A are not diagnostic but only suggestive. Thus, stringent and targeted analysis of ultrasound features of the fetus (typically in the second or third trimester) have been recommended to confirm or deny the diagnosis. However, even this diagnostic approach is of limited sensitivity and specificity because structural abnormalities found in fetuses with CdLS (e.g. nuchal translucencies) are observed in fetuses that carry other human developmental/genetic diseases, and because not all individuals with CdLS display structural abnormalities that may be detected by ultrasound.

Consequently, while some understanding of the genetic basis of CdLS has been gained, meaningful and economically sound diagnostic tests have not been developed. Therefore, there is still a need to provide improved composition and methods to improve genetic tests and develop therapeutic treatments for CdLS.

### SUMMARY OF THE INVENTION

The present invention is directed to kits, compositions, and methods for diagnosis and treatment of Cornelia de Lange Syndrome. More specifically, the inventors discovered that a NIPBL+/− (heterozygous) genotype is associated with changed expression of non-NIPBL genes, which may be monitored as surrogate markers for simplified and accurate diagnosis of CdLS. Such a test is also advantageous for the development of models that identify potential therapeutic drugs by monitoring increased/normalized expression of the NIPBL gene itself, and/or surrogate markers.

In one aspect of the inventive subject matter, a method of diagnosing CdLS includes a step of identifying a surrogate marker, wherein the surrogate marker is a gene that is over-expressed or under-expressed, relative to wildtype (normal) controls, in tissues/body fluids/DNA/mRNA taken from subjects of a known NIPBL+/− genotype. In another step, expression of the surrogate marker is measured to obtain a test result, and a person is then diagnosed as having the Cornelia de Lange syndrome using the test result.

Most typically, the NIPBL+/− genotype is caused by knockout, knockdown, or function-reducing mutation of at least one allele of the NIPBL gene in a non-human vertebrate, or by a mutation in at least one allele of the NIPBL gene, wherein the mutation is a mutation known to be associated with Cornelia de Lange syndrome. The surrogate marker is preferably a gene that is over-expressed or under-expressed at least 1.5 times, and more preferably at least 2.0 times as compared to expression of the same gene under the condition of a normal NIPBL+/+ genotype. Depending on the particular surrogate gene or genes, the step of measuring comprises multi-gene analysis (e.g., by gene chip analysis or quantitative PCR). Alternatively, the step of measuring may also comprise quantitative analysis of a gene product of the gene that is over-expressed or under-expressed. Most typically, contemplated methods will include analysis of more than one, more typically more than five surrogate, and most typically more than ten surrogate genes. Especially contemplated surrogate genes are listed in the attached sequence listing.

In another aspect of the inventive subject matter, a kit includes a genetically modified animal comprising a cell with NIPBL+/− genotype, and an instruction associated with the animal to use the animal as a model for Cornelia de Lange syndrome. The animal is most preferably a transgenic mouse in which one allele of the NIPBL gene is inactivated or eliminated, or in which at least one NIPBL allele has a mutation that is associated with Cornelia de Lange syndrome. It is further preferred that the cell with NIPBL+/− genotype is a cell selected from the group consisting of a neural cell, a blood cell (white or red), an hepatic cell, a heart cell, a bone (osteogenic) or bone marrow cell, a fat (adipose tissue) cell, a kidney cell, and/or a dermal cell. Thus, organ-specific as well as systemic NIPBL+/− genotypes are contemplated. In further preferred aspects of contemplated kits, the instruction comprises an information to measure expression of a surrogate marker, wherein the surrogate marker is a gene that is over-expressed or under-expressed as a function of a NIPBL+/− genotype (wherein the instructions may inform a user that measurement of the expression of the surrogate marker is performed after administration of a potential therapeutic agent).

Therefore, and viewed from yet another perspective, a method of identifying a diagnostic marker and/or therapeutic target for treatment of Cornelia de Lange syndrome may include a step of providing a recombinant or transgenic animal having a cell with NIPBL+/− genotype. In another step, a surrogate marker is identified, wherein the surrogate marker is a gene that is over-expressed or under-expressed as a function of a NIPBL+/− genotype, and in yet another, optional step, a potential therapeutic agent is administered to the animal. Where desirable, the change in expression of the surrogate marker may then be monitored after administration of the potential therapeutic agent (e.g., potential therapeutic agent increases expression of NIPBL or is a recombinant NIPBL gene or protein).

Various objects, features, aspects and advantages of the present invention will become more apparent from the following detailed description of preferred embodiments of the invention.

### BRIEF DESCRIPTION OF THE DRAWINGS

FIG. 1 is a graphic representation of selected murine genes that were identified as being up- or down-regulated in response to NIPBL+/− genotype.

FIG. 2 is a table listing human sequences homologous to those identified as being up- or down-regulated in a mouse model with NIPBL+/− genotype.

FIG. 3 depicts various aspects of bone development in Nipbl⁵⁶⁴/+ mice.

FIG. 4 depicts atrial septal defects in Nipbl⁵⁶⁴1+ mice.

FIG. 5 depicts craniofacial morphology of Nipbl⁵⁶⁴/+ mice.

FIG. 6 depicts auditory differences of Nipbl⁵⁶⁴1+ mice compared to wild type.

FIG. 7 illustrates cerebellar hypoplasia in Nipbl⁵⁶⁴/+ mice.

FIG. 8 depicts graphs showing reduced NIPBL expression in Nipbl⁵⁶⁴/+ mice.

### DETAILED DESCRIPTION

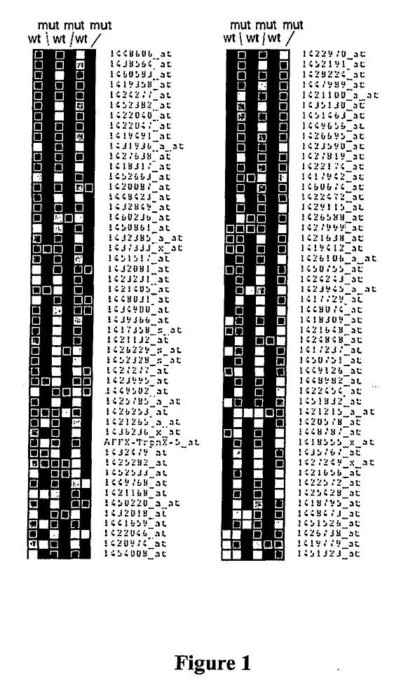

The inventors have now discovered that functional and/or quantitative changes in the expression of the NIPBL gene are also associated with changes of expression in other genes, which may be monitored as surrogate markers for simplified and accurate diagnosis of CdLS. Most preferably, surrogate markers will be measured from peripheral blood, but may also be determined in amniotic fluid, urine, cells cultured from biopsies, or solid tissues. It should also be appreciated that such measurement may involve measurement of expression of surrogate genes as well as their expression products. FIG. 1 depicts an exemplary heat map of an Affymetrix gene chip indicating increased and decreased expression of selected murine genes on the chip as a function of NIPBL+/− genotype (see also below).

Particularly contemplated changes of expression of NIPBL include up- and/or down-regulation of transcription and/or translation of the NIPBL gene, changes in the quantitative balance of splice variants, conformational changes of transcripts or the protein (e.g., alternate secondary structures in unprocessed transcripts), and/or informational changes in the NIPBL-gene (e.g., transition, transversion, translocation, insertion, deletion, etc.), all of which may or may not lead to functional alteration of the expressed NIPBL gene product, loss or addition in glycosylation sites, etc.

As used herein, the term “NIPBL+/− genotype” refers to a genotype in which one allele is a wild type allele (or has silent mutation) and in which the other allele is eliminated or muted (e.g., via knockout or gene trap), or functionally impaired (e.g., using known mutations that produce clinical symptoms characteristic of CdLS). The term “NIPBL+/− genotype” also includes genotypes in which transcription and/or translation of at least one allele is reduced (e.g., knockdown or RNA interference). Furthermore, as also used herein, the term “NIPBL” refers to the human “Nipped-B homolog (Drosophila)” and all functional gene homologs that encode the NIPBL protein (also known as “delangin”) and homologous proteins. Consequently, the term NIPBL includes NIPBL, NIPBL, Nipped-B, Nipbl and Nipbl.

It should be appreciated that there are numerous manners of generating a “NIPBL+/− genotype” known in the art, and all of the known manners are deemed suitable for use herein. For example, a NIPBL+/− genotype can be achieved using site-specific insertional mutagenesis, which may or may not be made reversible (e.g., Cre/loxP-induced conditional knockout system) and/or inducible (e.g. tamoxifen-inducible, tet on-off systems, etc.). Alternatively, random insertion and selection for suitable mutants (e.g., via gene trap methods) may provide the desired NIPBL+/− genotype. Regardless of the manner of forming the NIPBL+/− genotype, it is preferred that the NIPBL+/− genotype is expressed systemically. However, the inventors also contemplate a tissue specific NIPBL+/− genotype via controlled expression in selected tissues ((e.g., by tissue-specific gene knockout or knockdown using tissue-specific promoters). With respect to the particular origin of contemplated NIPBL gene, it should be appreciated that all sources (typically, but not necessarily mammalian) are deemed suitable herein, and the particular host cell/tissue/animal may at least in part determine the choice of the NIPBL gene.

In especially preferred aspects of the inventive subject matter, the NIPBL+/− genotype is generated in a transgenic, transformed, antisense RNA-treated, or double-stranded RNA-treated animal, more preferably a vertebrate, and most preferably a mouse or non-human primate. Particularly contemplated animals include rodents (e.g., mouse, rat, etc.), fish (e.g. Danio rerio, a.k.a. zebrafish), various yeasts (e.g., Saccharomyces, Pichia, etc.), invertebrates (e.g., Caenorhabditis, Drosophila, etc.), and monkeys and apes (e.g., rhesus, chimpanzee). It should be noted, however, that the NIPBL+/− genotype may also be generated in an isolated organ (e.g., transformed skin, muscle, or liver), isolated tissue (e.g., transformed neural, hepatic, embryonic), or cell culture (e.g., transformed fibroblast, hepatocyte), wherein suitable organs, tissues, or cells may be derived from various sources, particularly from the animal sources listed above.

Once the desired NIPBL+/− genotype has been established in a suitable carrier (e.g., animal, organ, tissue, or cell culture) to generate a test system, the process of identifying non-NIPBL genes and/or gene products that exhibit a change in expression as a function of the NIPBL+/− genotype (i.e., the surrogate marker) can be performed using numerous methods well known in the art. The term “over-expressed or under-expressed as a function of a NIPBL+/− genotype” as used herein refers to a statistically significant over- or under-expression of a gene in a cell, tissue, organ, or animal having NIPBL+/− genotype as compared to the level of the gene measured under control conditions in a cell, tissue, organ, or animal having NIPBL+/+ genotype (diagnosed as not having CdLS). To that end, one or more non-NIPBL genes and/or gene products are identified and/or selected, and their expression is monitored in an animal, organ, tissue, or cell culture having NIPBL+/− genotype as well as in a control (normal, wildtype) animal, organ, tissue, or cell culture under otherwise identical experimental conditions.

Differences in expression can then be monitored using all manners known in the art. For example, differentially expressed genes may be identified using a gene chip for selected groups of genes, randomly selected genes, or (comprehensive) gene panels. Alternatively, array PCR, subtractive hybridization, and other differential techniques may be employed to identify genes that are over-expressed or under-expressed under condition of the NIPBL+/− genotype. Where the expression difference is observed by monitoring gene products, analysis may be done by two dimensional electrophoresis, immunoblotting, mass spectroscopy, etc. Alternatively, all other known manners of quantifying expression products are also deemed suitable. In preferred aspects, more than one, more typically more than three, even more typically more than five, and most typically more than ten surrogate markers are measured. Most preferably, thus identified surrogate markers will have a difference in expression of at least ±0.5-fold, more typically at least ±0.7-fold, even more typically at least ±1.2-fold, and most typically at least ±1.5 to ±2.0-fold, wherein the difference is preferably deemed statistically significant (e.g., with p<0.1). Such quantitative analysis is well known in the art and can be performed by the person of ordinary skill in the art without undue experimentation.

Identified surrogate markers may then be grouped by clinical significance, quantitative strength in expression level, cellular and/or tissue origin, etc., and more simplified assay procedures may be established. For example, surrogate markers identified from various gene chips may be quantified individually using other hybridization methods or quantitative PCR protocols, or where the marker is an enzyme, quantification may be performed using a colorimetric assay.

Consequently, it should be appreciated that a method of diagnosing a person as having Cornelia de Lange syndrome may include a step of identifying a surrogate marker, wherein the identification may be performed on a case-by-case basis, or more typically, by reference to an already established and known set of surrogate markers which may or may not come from the same species (e.g., human ortholog of a murine gene). Most preferably, the surrogate marker is a gene or gene product that is over-expressed or under-expressed as a function of a NIPBL+/− genotype. In a still further step, a patient sample is obtained (e.g., whole blood by venipuncture, cells by buccal swab, amniocentesis, or tissue by biopsy, including chorion villi biopsy) and expression of the surrogate marker is then measured to obtain a test result. With respect to the person, it should be appreciated that the term “person” includes a post-natal person as well as an embryo and a fetus. Depending on the particular type of surrogate marker it should be appreciated that the measurement may be direct (e.g., quantitative rtPCR or hybridization) or indirect (e.g., via colorimetric assay), and all known manners of quantification of levels and/or activities of a specific nucleic acid and/or protein are deemed suitable for use herein. Based on the test result, the person is then diagnosed as having Cornelia de Lange syndrome. For example, a positive diagnosis may be provided where at least two, more typically at least five, and most typically at least ten surrogate markers are over-expressed and/or under-expressed at least 0.7 times, more typically 1.0 times, even more typically at least 1.5 times, and most typically at least 2.0 times as compared to expression under NIPBL+/+genotype (i.e., genotype of a person not affected with CdLS).

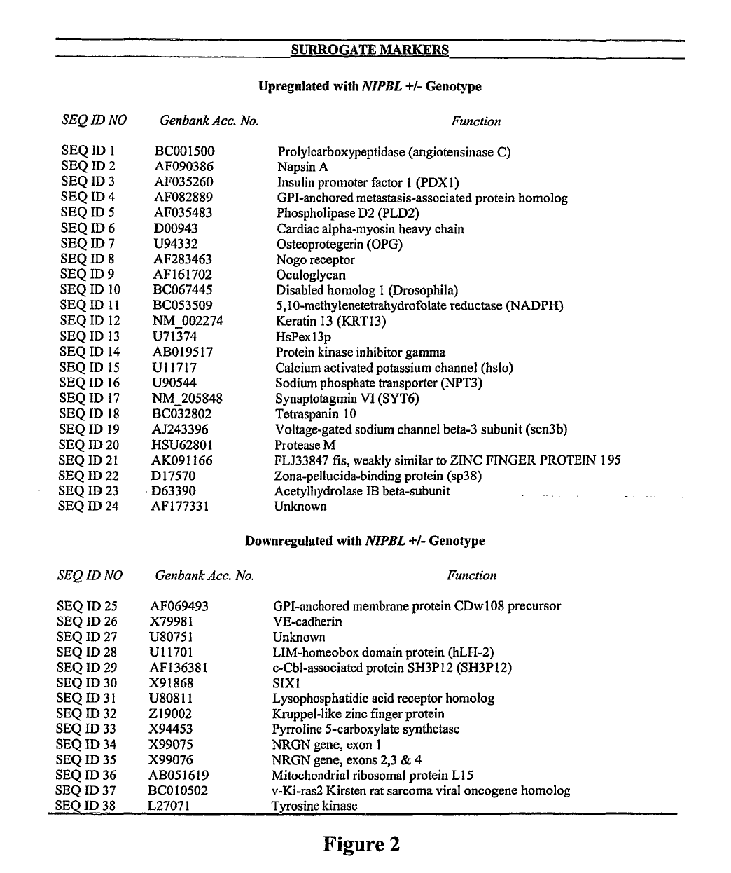

With respect to the surrogate markers, it is generally contemplated that the associated changes in expression will include up- and/or down-regulation of transcription and/or translation of the surrogate genes/gene products. Moreover, as gene expression is frequently coordinated with expression of other genes, cascades of genes may be measured that may functionally and/or developmentally be associated with each other. Of course, it should be appreciated that the surrogate markers may be detected as nucleic acids (e.g., mRNA via qPCR) and/or as polypeptides, which may be directly detected (e.g., using antibody labeling) or indirectly by virtue of their structure and/or function (e.g., enzymatic activities) as discussed above. Moreover, it should be recognized that contemplated surrogate markers may be obtained from homologous systems (e.g., human markers from human cell culture with NIPBL+/− genotype) or from heterologous systems (e.g., human markers from murine animal model having NIPBL+/− genotype). Thus, genes identified as being affected by the NIPBL+/− genotype may be directly used as a surrogate markers, or may be used to select an ortholog or homolog sequence. For example, genes that were identified in a mouse model (substantially as described below) were used to identify human ortholog sequences, which may then be employed as surrogate markers for diagnosis and/or therapy. Particularly preferred human ortholog sequences (SEQ ID NO 1-38) are listed in the table of FIG. 2 and are identified by corresponding Genbank accession number and actual/tentative function. Preferred genes will exhibit up- or down-regulation of expression in an amount of at least 1.5-fold, more typically at least 1.7-fold, and most preferably 2.0-fold.

Depending on the particular marker or markers, it should be noted that the diagnostic test may involve one or more methods. However, it is typically preferred that the test will be in an automatable format, and most preferably include biological fluids (e.g., blood, serum, urine, etc) as sample material. Therefore, especially suitable test methods include qualitative and quantitative methods of nucleic acid detection (e.g., PCR-based methods, SNP detection, etc.), qualitative and quantitative methods of protein detection (e.g., using labeled antibodies), qualitative and quantitative methods of measuring protein activity (e.g. assays of enzyme function), cell based methods (e.g., using FACS), and preparative qualitative and quantitative analytical methods (e.g., using HPLC, LC, GC, MS, etc.) well known in the art.

Furthermore, depending on the particular correlation of a particular mutation in the NIPBL gene and the expression of the associated surrogate gene, it should be recognized that the surrogate marker(s) may not only be employed to confirm CdLS diagnosis, but may also be used to grade the severity of the CdLS. Additionally, and especially where multiple markers are tested, it is also contemplated that a particular marker pattern may be predictive of a particular NIPBL mutation. Such indirect “fine-tuned” diagnosis may then be useful to provide proper treatment and follow-up to a patient.

Therefore, in another aspect of the inventive subject matter, a kit is contemplated that includes a genetically modified animal comprising a cell with NIPBL+/− genotype. Such kit will further be associated (e.g., via print, website, or otherwise provided and making reference to the animal) with an instruction to use the animal as a model for Cornelia de Lange syndrome. For example, mice defective in one allele of the NIPBL gene can be prepared using genetically modified mouse embryonic stem cells (e.g., gene trap modification using alternate splice acceptor site and termination pA signal). Such mice may be confirmed to have the NIPBL+/− genotype by methods well known in the art (e.g. genomic PCR for the gene trap modification). In such mice, levels of expression of genes can then be measured (e.g. by oligonucleotide hybridization or quantitative RT-PCR) in samples derived from body fluids or tissues such as liver. Where desirable, the animal model may also include reference to already identified surrogate markers, wherein the markers may or may not be specific to a particular tissue (e.g., liver, fatty tissue, neural tissue, cardiac tissue, fibroblasts, lymphocytes, etc.), or may instruct a user to measure expression of a surrogate marker, wherein the surrogate marker is a gene that is over-expressed or under-expressed as a function of a NIPBL+/− genotype (e.g., in a neural cell, a hepatic cell, a blood cell, a heart cell, a bone cell, a bone marrow cell, an adipose cell, and/or a dermal cell).

Alternatively, and as already described above, the NIPBL+/− genotype animal model need not be restricted to murine models, but may use various vertebrates and invertebrates. For example, where high number of progeny and low generational age is desired, insect or nematode based models, or even cellular models may be employed. On the other hand, where similarity to human expression patterns is desired, vertebrate models or even transformed human cell and tissue cultures may be employed.

In still further contemplated aspects, the so identified surrogate markers may also be employed as therapeutic targets. Therefore, a method of identifying a diagnostic marker or therapeutic target for treatment of Cornelia de Lange syndrome may include a step of using or providing an animal model according to the inventive subject matter. In another step, one or more surrogate markers are identified, wherein the surrogate marker(s) is/are a gene that is over-expressed or under-expressed as a function of a NIPBL+/− genotype. Based on the so obtained knowledge, a potential therapeutic agent can then be administered to the animal (or cell or tissue culture) and a change in expression of the surrogate marker may be measured after administration of the potential therapeutic agent to thereby identify corrective action (which can be asserted by comparison with NIPBL wild-type models).

For example, where a deficiency in the surrogate marker caused by a change in the NIPBL gene leads or contributes to CdLS, functional complementation is particularly preferred. Such complementation may be performed using administration of the surrogate marker, by supplementing the patient with a product produced by the surrogate marker, or by removing a substrate or product from the patient (e.g., via diet) that is removed/produced by the surrogate marker. Alternatively, recombinant gene therapy may be used to correct the quantities of the surrogate markers towards normal levels observed in human not affected with CdLS. On the other hand, and especially where the surrogate marker is present in excess, inhibitors or antibodies against the marker may be administered. Thus, it should be recognized that the animal models presented herein may not only be employed to develop a test for diagnosis, but may also be used as a platform to develop suitable treatment options for a patient diagnosed with CdLS. For example, upregulated surrogate markers may be suitable targets for inhibitors, especially where such targets are enzymes. Similarly, downregulated surrogate markers may indicate that at least some treatment success may be possible by supplementation and/or complementation with a metabolite or enzyme. Therefore, potential therapeutic targets may also be useful for improving clinical signs where a patient is diagnosed with CdLS.

Alternatively, and even more preferably, it is contemplated that intact allele of the NIPBL gene in a person diagnosed with CdLS may be employed as a therapeutic target. In such an approach, strategies could be identified, using an animal or cell based model, whereby expression of the NIPBL wild-type allele (which is present in virtually all affected individuals) can be increased to a degree that at least partially compensates for the reduced or absent function of the mutant, NIPBL− allele.

It should be recognized that such an approach is especially conceptually attractive because CdLS appears to follow the pattern of a haploinsufficiency disease (with respect to NIPBL). Moreover, as discussed in the following description of experimental results, the NIPBL gene appears to be under autoregulatory control, which implies that mechanisms, presumably involving other gene products such as transcription factors and signaling proteins, already regulate NIPBL expression, and therefore comprise potential targets for therapeutically useful pharmacological intervention.

The presence of one normally functional NIPBL allele, and the compensatory upregulation of that allele, in individuals with CdLS, as well as in animals with a NIPBL+/− genotype, together imply that the amount of increase in NIPBL expression that would be required to restore fully normal function is small, on the order of 1.3 to 1.5-fold. Ultimately, this is encouraging, insofar as only small pharmacological effects may be needed to produce such a change. However, finding such therapeutic agents by screening through libraries of components or other large sets of substances will be very difficult if measurement of NIPBL level is used as the assay endpoint, since differences in transcript levels of 1.3-1.5 fold are not often within the accuracy of assays commonly used in the art for large scale screening. A more practical approach, which is provided by the compositions and methods presented herein, involves using as the assay endpoint for therapeutic screening and validation, the levels of surrogate gene markers for the NIPBL+/− genotype, some of which increase or decrease by 2-fold or more under the NIPBL+/− condition. Not only is the signal-to-noise ratio of therapeutic screening and validation improved by using surrogate markers rather than NIPBL levels, but specificity is improved by the greater statistical power of using multiple surrogate markers at once. Thus, it should be appreciated that contemplated kits and methods provide a leveraged analytical tool to observe boosted NIBPL expression in an especially practical and effective way.

### Experiments

The following experiments illustrate an exemplary animal model for CdLS and data in support that such animal model closely paralleles genetic, physiologic, developmental, and morphologic characteristics of human CdLS. In these experiments, mice heterozygous for a gene-trap mutation in NIPBL exhibit multi-system defects characteristic of CdLS, including small size, microbrachycephaly, heart defects, hearing abnormalities, and delayed bone maturation. The presence of additional abnormalities is evidenced by a high (˜75%) incidence of perinatal mortality. Interestingly, these phenotypes are associated with a decrease in NIPBL transcripts of only 25-30%, implying an extreme sensitivity of developmental events to small changes in NIPBL function.

### Materials and Methods

A search for NIPBL sequences in publicly available mouse gene trap databases (http://www.genetrap.org/) initially identified two targeted ES cell lines. One of these (RRS564) contains a gene trap in intron 1, upstream of the first coding exon; the other (RRJ102) is in intron 25 (the human annotation of Krantz et al. (Nat Genet (2004) 36, 631-635) is used here for exon numbering). Both cell lines were obtained and injected into blastocysts of C57B1/6 mice. Multiple male chimeras were obtained from each line, and were bred against outbred (CD-1) mice. Germ line transmission was detected by the chinchilla coat color of ES-derived progeny. Germ line progeny were obtained only from RRS564-derived chimeras. Unless indicated otherwise, the RRS564 gene trap allele is hereinafter referred to as Nipbl564 and mice heterozygous for this allele as Nipbl⁵⁶⁴/+. Nipbl⁵⁶⁴/+ mice were maintained under normal laboratory conditions, and the line perpetuated by successive rounds of breeding to CD-1 mice. As already indicated earlier, it should be noted that the Nipbl⁵⁶⁴/+ is a particular (and non-limiting) example of an NIPBL+/− genotype, and it should be recognized that numerous other mutations in the Nipbl gene will produce the same effects as further reported below.

Anatomical and histological evaluation was performed using fresh-frozen or paraformaldehyde fixed tissues. In some cases, fixation was carried out by intracardiac perfusion. Alcian Blue/Alizarin Red staining was carried out using known methods. Auditory brainstem response recordings were generated as described by Zheng et al. (Hearing research (1999) 130, 94-107). Briefly, a cohort of young adult mutant and littermate control animals were anesthetized and surface electrodes used to detect brainstem responses to a variety of clicks and tones introduced into one ear.

Measurements of Nipbl levels by RNase protection were made according to standard methods. Briefly, for each reaction, 20 μg of total RNA was hybridized with ³²P-labeled probes for Nipbl (containing 39 bases of exon 10, all 183 bases of exon 11, and 4 bases of exon 12; 90,000 cpm) and Gapdh (containing 116 bases of exon 4 and 15 bases of exon 5; 20,000 cpm) and processed according to manufacturers instructions (Ambion RPA III kit). Samples were run on a 5% polyacrylamide/8M urea gel, dried, and bands quantified by phosphorimager.

Microarray analysis of gene expression (Golub et al., Science (1999) 286, 531-537) in Nipbl⁵⁶⁴/+ and wildtype mice was performed on total RNA isolated from whole liver. RNA was labeled and hybridized to Affymetrix Murine 430 2.0 array chips using the protocol described at http://www.broad.mit.edu/mpr/publications/projects/Leukemia/protocol.html, and data were analyzed using GenePattern software (http://www.broad.mit.edu/cancer/software/genepattern). Genes that were substantially up- or downregulated in Nipbl⁵⁶⁴/+ liver for each of three sex-matched littermate pairs were identified as being potential Nipbl-sensitive targets.

### Results

### Heterozygous Mutation of Nipbl is Associated with Perinatal Mortality

Among the germline progeny of RRS564 chimeric mice, Mendelian inheritance predicts that 50% should be Nipbl⁵⁶⁴/+. However, when chinchilla offspring of such chimeras were assessed at weaning (3 weeks), the mutant allele was found in only 22%, as shown in Table 1. These animals (“N₀” generation) were fertile, and when bred against CD-1 females, only 18% of their surviving progeny carried the mutant allele. When animals of this “N₁” generation were again crossed to CD-1 females, again only 18% of their surviving progeny carried the mutant allele.

TABLE 1
Heterozygosity for Nipbl⁵⁶⁴ is associated with marked lethality

| | | | | Viable at | | | Resorption at | |
|---|---|---|---|---|---|---|---|---|
| Paternal | Surviving >3 weeks | | | E17.5-E18.5 | | | E17.5 | |
| genotype | +/+ | +/− | ratio | +/+ | +/− | ratio | +/+ | +/− |
| Chimera | 79* | 22* | 3.6:1^{‡} | nd | nd | nd | nd | nd |
| N₀ Nipbl⁵⁶⁴/+ | 159 | 35 | 4.5:1^{‡} | 37 | 30 | 1.2:1 | 0 | 2 |
| N₁ Nipbl⁵⁶⁴/+ | 28 | 6 | 4.7:1^{#} | nd | nd | nd | nd | nd |

*Only animals with chinchilla coats scored.
^{‡}P < 0.001 by chi-squared analysis
^{#}P < 0.02 by chi-squared analysis
^{†}P = 0.67 to Mendelian and <0.005 to postnatal distribution

The data imply that ˜75% of heterozygous Nipbl⁵⁶⁴+ mutants die before weaning, and that this fraction remains stable as the Nipbl⁵⁶⁴/+ allele is crossed off its original genetic background. Although postnatal animals were generally not genotyped until after weaning, litter sizes were assessed the morning following birth, and generally did not decline thereafter. Furthermore, when litters were first assessed, dead or dying neonates were observed no more frequently than in wildtype litters (not shown). These data suggest that lethality occurs either during the embryonic period or shortly enough after birth that maternal cannibalism occurs before litters can be assessed.

To distinguish among these possibilities, the inventors examined the frequency of Nipbl⁵⁶⁴/+ mutants shortly before birth (gestational day 17.5-18.5). Because no visible marker was available for identifying the ES-cell derived progeny in litters fathered by chimeras, this test was carried out with litters fathered by the N₀ generation. As shown in Table 1 above, mutants accounted for 41% of all progeny, a frequency not statistically significantly different from the expected Mendelian frequency of 50%. Although one cannot rule out the possibility of a small degree of prenatal lethality, it is clear that most Nipbl⁵⁶⁴/+ mutants die at or just after birth.

To investigate this further, offspring of an N₀ mutant father were delivered at term (embryonic day 18.5) by Caesarian section. Of 14 littermates, seven wildtype animals commenced regular breathing following brief physical stimulation, and within 15 minutes all displayed a pattern of regular shallow breathing and frequent spontaneous limb movements. In contrast, all seven mutant embryos exhibited difficulty with revival: Although all responded to stimulation with gasping movements, two never breathed spontaneously and ultimately became unresponsive and expired (upon subsequent dissection no air was observed in the lungs of these animals). Of the remaining five, two commenced spontaneous breathing later than their wildtype littermates, and three were still breathing irregularly 40 minutes after birth and showed little limb movement until nearly an hour had elapsed. These data strongly suggest that compromised cardiac or pulmonary function at birth accounts for perinatal lethality in Nipbl⁵⁶⁴/+ mutant mice.

### Nipbl⁵⁶⁴/+ Embryos are Growth-Retarded and Exhibit Delayed Bone Maturation

Among the most commonly observed clinical features of CdLS are small body size evident well before birth, and abnormalities of the upper limbs, ranging from small hands to frank limb reductions. As shown in Table 2 below, the inventors observed that Nipbl⁵⁶⁴/+ embryos at days 17.5 and 18.5 of gestation were 18-19% smaller than their wildtype littermates (P<0.001). This reduction was not accompanied by a reduction in placental size. Nipbl⁵⁶⁴/+ embryos at earlier stages were also smaller than littermates, but insufficient data were collected for statistical analysis.

TABLE 2
Prenatal growth retardation in Nipbl⁵⁶⁴/+ embryos

| | E17.5 | | | E17.5 | | | E18.5 | | |
|---|---|---|---|---|---|---|---|---|---|
| | Body weight (g) | | | Placental weight (g) | | | Body weight (g) | | |
| Genotype | Mean | S.D. | N | Mean | S.D. | N | Mean | S.D. | N |
| +/+ | 1.02 | 0.096 | 30 | 0.115 | 0.015 | 23 | 1.27 | 0.076 | 7 |
| Nipbl⁵⁶⁴/+ | 0.80^{#} | 0.115 | 23 | 0.112^{†} | 0.024 | 17 | 1.00^{#} | 0.082 | 7 |

Embryos were dissected at the indicated times from crosses of N₀ Nipbl⁵⁶⁴/+ males and CD-1 females. Growth retardation of 18-19% is apparent at both ages, without a significant difference in placental weight.
^{#}P < 0.001,
^{†}P = 0.64, by Student's t-test

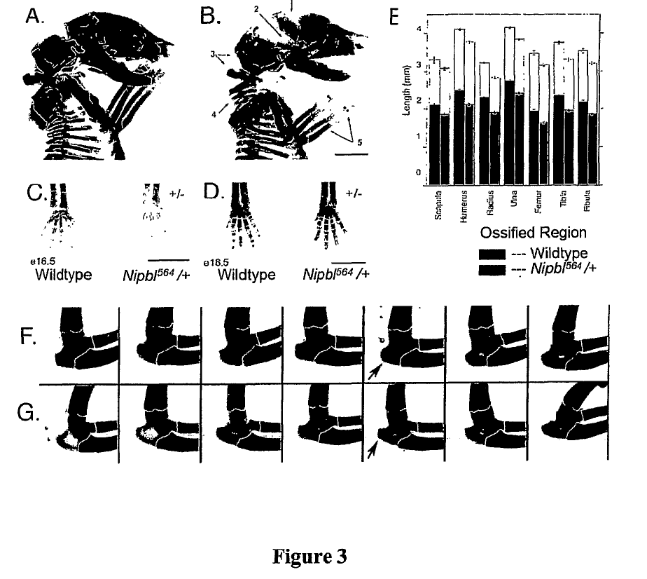

Nipbl⁵⁶⁴+ embryos did not display limb or digit truncations, or loss of any other bony elements, findings that can be observed in a subset of individuals with CdLS. However, upon staining of embryonic skeletons using Alcian Blue and Alizarin Red, the inventors observed noticeable delays in ossification of both endochondral and membranous bones of Nipbl⁵⁶⁴/+ embryos. As shown in FIG. 3A-D, delayed ossification of the skull and digits was readily apparent between E16.5 and E18.5. Measurement of long bones and digits at E17.5 revealed, in addition to a symmetrical reduction in bone length (consistent with the overall difference in body size), a statistically significant decrease in the relative extent of ossification (FIG. 3E). Otherwise, the patterning of cartilaginous elements was relatively normal, although some subtle differences in morphology were appreciated, e.g. in the shape of the olecranon process (FIGS. 3F-1G).

FIG. 3 generally depicts bone development in Nipbl⁵⁶⁴/+ mice. More specifically, Alcian Blue/Alizarin Red staining was used to analyze Nipbl⁵⁶⁴/+ embryos and wildtype littermates at embryonic days 16.5, 17.5 and 18.5. This technique stains cartilage blue and bone red. In FIG. 3, blue staining appears as light grey and red staining as dark grey or black. A-B. cranial and trunk skeleton at E17.5. A. Wildtype and B. Nipbl⁵⁶⁴/+. Arrows indicate locations at which bone development appears substantially delayed in the mutant. C. Forepaws at E16.5 Skeletal elements are patterned normally in the mutant, but are smaller than in wildtype litter mates. D. Forepaws at E18.5. Delayed ossification is readily seen in the mutant metacarpals and phalanges. E. Measurements of long bone lengths and degree of ossification at E18.5. For each bone listed, the left bar represents the wildtype measurement, and the right the mutant measurement, averaged over >9 independent measurements in each case. The shaded portion of each bar represents the ossified region of each bone. Note that bones of mutant animals are about 10% shorter than wildtype. In addition, the percent of bone length that is ossified is 5-7% less in mutants. F-G. Higher magnification view of the elbow joint of seven wildtype (panel F) and seven mutant (panel G) embryos at E18.5. Notice how the olecranon process (arrow) is longer and appears to come to a sharper point in the majority of mutant embryos.

### Nipbl⁵⁶⁴/+ Embryos Exhibit Cardiac Septal Defects

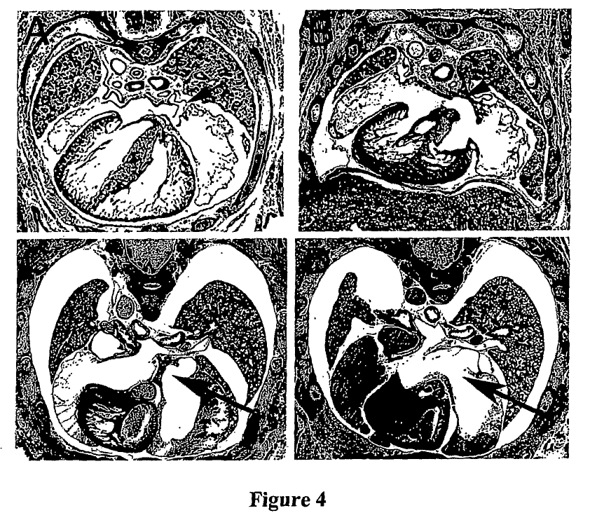

Cardiac defects occur commonly in CdLS, especially atrial and ventricular septal defects (Am J Med Genet (1993) 47, 940-946; Am J Med Genet (1997) 71, 434-435; Am J Med Genet (1998) 75, 441-442). Among Nipbl⁵⁶⁴/+ mouse embryos, obvious atrial septal defects were observed by the inventors in about 50% of cases. Such defects were typically large as evidenced in FIG. 4, and could be detected as early as embryonic day 15.5, shortly after atrial septation normally finishes. FIG. 4A-B depict abnormalities of cardiac development in Nipbl⁵⁶⁴/+ mice at embryonic day 15.5, when atrial septation is normally finishing. The septum primum and septum secundum are readily apparent in wild type heart, (A, arrow) but are reduced in the Nipbl⁵⁶⁴/+ embryo (B). At E17.5, a well-formed atrial septum is apparent in the wildtype heart (C, arrow), but is absent in the mutant (D) A noticeable reduction in atrial size was also seen in some mutant animals, but this was not a consistent finding.

No consistent defects were detected in the atrioventricular valves or septum, outflow tract, or pulmonary vasculature. However, many mutant embryos displayed subtle abnormalities of the ventricular and interventricular myocardium, including abnormal lacunar structures and generalized disorganization of the compact layer, especially near the apex (data not shown). Interestingly, no cardiac abnormalities were detected among mutant animals that survived birth, whether at postnatal day 0, or in adulthood. This implies that that the cause of high perinatal mortality is either cardiac, or correlates strongly with the presence of cardiac structural defects.

Histological examination of other organ systems in late embryonic Nipbl⁵⁶⁴/+ mutant mice revealed no obvious anatomical abnormalities of the lungs, diaphragm, liver, stomach, spleen, kidney or bladder. Brains of embryonic and neonatal Nipbl⁵⁶⁴/+ mice displayed relatively normal gross anatomy, although a single mutant was observed to have a large brainstem epidermoid cyst (data not shown).

### Surviving Nipbl⁵⁶⁴/+ Mice are Small and Lean

Those Nipbl⁵⁶⁴/+ mice that survived birth generally survived to adulthood, and many have been maintained for over a year. Interestingly, the ˜20% weight difference between mutant and wildtype animals at birth became exacerbated during the first 3-4 weeks of postnatal life, so that mutants weighed only 50-60% of sex-matched wild type levels at weaning. Subsequently, Nipbl⁵⁶⁴/+ mice exhibited rapid catch-up growth, reaching 65-70% of wildtype weight by 6-7 weeks, the normal age of sexual maturity. This finding raises the interesting possibility that, in addition to being intrinsically small, mutant animals may also receive inadequate nutrition when suckling. Interestingly, according to published data (Am J Med Genet (1993) 47, 1042-1049), the weights of children with CdLS also fall further behind age norms during the first year of life, then show significant but incomplete catch-up later on.

As is typical of laboratory mice fed ad libitum, the wildtype littermates of Nipbl⁵⁶⁴/+ mice continue to gain weight after maturity, especially males, who usually accumulate large amounts of body fat. Interestingly, the weights of mature Nipbl⁵⁶⁴/+ mice increased at a much slower rate, suggesting that mutant animals stay relatively lean. Indeed, dissections revealed that the abdominal contents of older male Nipbl⁵⁶⁴/+rarely contained the large collections of visceral fat so common in their wildtype littermates.

### Nipbl⁵⁶⁴+ Mice have a Distinctive Craniofacial Morphology

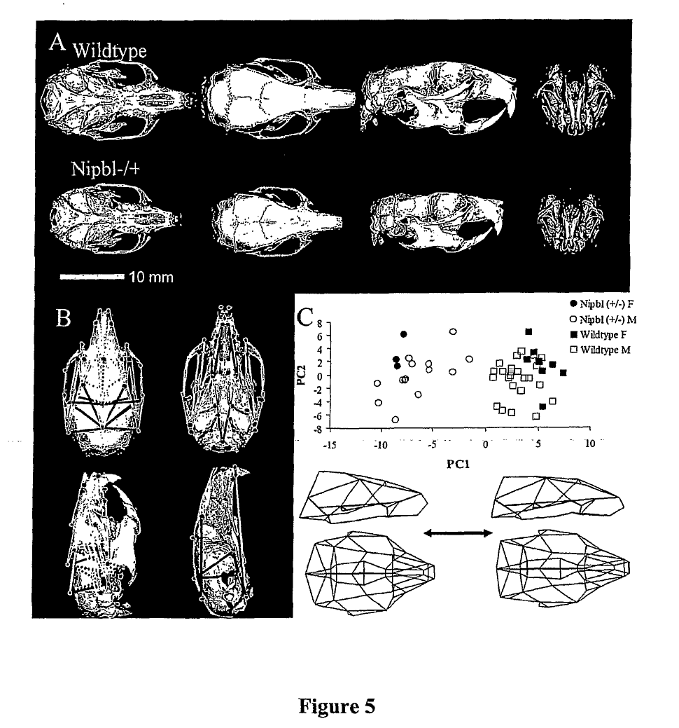

The craniofacial features of CdLS are highly characteristic and include microbrachycephaly, synophrys, upturned nose and down-turned lips. Micro-CT analysis of adult Nipbl⁵⁶⁴/+ and wildtype littermate skulls showed a reduction in cranial volume consistent with reduced body size. In addition, mutant animals also displayed a disproportionate foreshortening of the anterior-posterior dimensions of the skull (i.e. brachycephaly; FIG. 5B), and an upward deflection of the tip of the snout (FIG. 5C and data not shown).

FIG. 5 depicts the craniofacial morphology of Nipbl⁵⁶⁴/+ mice. A. Representative reconstructions of wildtype and Nipbl⁵⁶⁴/+ skulls based on microCT scans. B. Results of Euclidean Distance Matrix Shape analysis. Distances shown are those that differ by more than 5% between the groups. The two groups are significantly different in shape by a Monte Carlo randomization test (p<0.001). Gray lines are distances that are relatively smaller in the mutant, while black lines are those that are relatively larger. C. Results of Procrustes based analysis for overall shape variation. The two groups are significantly different in shape by Goodall's F-test (p<0.001). The first principal component for the combined sample captures the shape variation that distinguishes the groups. This shape variation is shown in the wireframe diagrams below the scatterplot. Canonical variates analysis for the shape differences between the sample yielded practically identical results.

### Nipbl⁵⁶⁴+ Mice Exhibit Hearing Deficits and Cerebellar Hypoplasia

Numerous neurological abnormalities are seen in CdLS, including mental retardation, abnormal sensitivity to pain, and seizures. In addition, some degree of hearing loss is observed in almost all individuals with CdLS, and this may play a role in some of the speech difficulties seen in this syndrome.

In Nipbl⁵⁶⁴/+ mice, hearing was assessed by auditory brainstem evoked responses (ABR). ABR abnormalities were observed in the majority of Nipbl⁵⁶⁴/+ mice tested. In a few cases, markedly increased thresholds to stimulation were observed. More commonly, stimulus thresholds were within normal limits, but the relative intensities of the components of the ABR were altered. In particular, mutant mice displayed a highly characteristic reduction in the amplitude of the third peak (at about 3 msec following the stimulus), a latency consistent with an abnormality within the inner ear or early brainstem neural pathways.

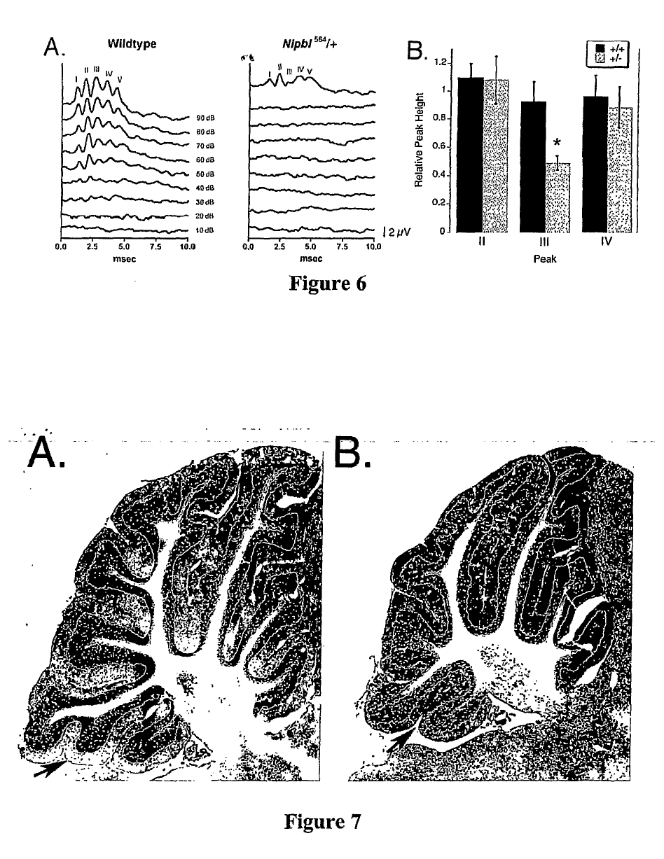

FIG. 6 depicts hearing deficits of Nipbl⁵⁶⁴/+ mice compared to wild type as measured by ABR. A. ABR records for a pair of wildtype and Nipbl⁵⁶⁴/+ littermates. Five distinct peaks are detected in the responses of animals to a pure tone stimulus, each with a characteristic latency and an amplitude that grows with stimulus intensity (each curve represents a 10 dB increment). A marked increase in threshold to stimulation indicates a dramatic hearing deficit in this mutant animal. Such a threshold increase is seen in less than half of mutant animals. B. Average background-subtracted sizes of Peaks II, III, and IV (normalized to Peak I to correct for experimental variation in amplitudes among different animals due to electrode placement) for the 90 dB tone response of the six wildtype and six mutant animals. Notice the marked depression of peak III in mutant animals (P<0.02). These data strongly suggest that the majority of Nipbl⁵⁶⁴/+ mice exhibit brainstem abnormalities in the auditory pathway.

FIG. 7 illustrates cerebellar hypoplasia in Nipbl⁵⁶⁴/+ mice. Adult wildtype and Nipbl⁵⁶⁴/+ mice were sacrificed by intracardiac perfusion with paraformaldehyde, and brains were removed, weighed, postfixed, embedded and cyrostat-sectioned. Approximately midsaggital sections through the cerebella of a wildtype (A) and mutant (B) littermate (N₁ generation; age=80 days). Note the smaller overall cerebellar size, with less well developed foliation, in the mutant (arrows). The brain weights of animals A and B were 0.65 g and 0.43 g respectively. Total cerebellar areas for these sections were 8.16 mm² and 5.715 mm², respectively. Based on these areas, the predicted decrease in cerebellar volume in the mutant is 42%, which is greater than the 34% reduction in total brain weight. This suggests that cerebellar development is especially sensitive to reduced Nipbl function. Although Nipbl⁵⁶⁴/+ mice were not subjected to other neurological or behavioral tests, it was noted that several animals exhibited long-term, repetitive circling behaviors. Others were noted to adopt opisthotonic postures in response to administration of anesthetics (suggestive of anesthetic-induced seizures).

The inventors detected no gross abnormalities in brain anatomy in adult Nipbl⁵⁶⁴/+ mice besides the cerebellar hypoplasia shown in FIG. 7. Similar findings, particularly in the midline the cerebellum, are among the few consistently reported changes in brain anatomy found in CdLS.

### Nipbl⁵⁶⁴/+ Mice Develop Corneal Opacities

Children with CdLS display a range of opthalmological abnormalities including ptosis, myopia, nasolacrymal duct obstruction, strabismus and blepharitis (J Pediatr Opthalmol Strabismus (1990) 27, 94-102; Archives of opthalmology (2006) 124, 552-557; J Aapos (2005) 9, 407-415). The inventors noticed that a subset of Nipbl⁵⁶⁴/+ mice (8/63 examined) developed various degrees of ocular opacification (not shown), often beginning as early as 3 weeks of age (no such changes were seen in 140 littermate controls). In several cases this condition became associated with periorbital inflammation and permanent closure of the eyelids. Histological analysis showed inflammatory and fibrotic changes within the cornea, consistent with repeated abrasion or injury. Such injury might arise from neglect, due to abnormalities in corneal sensation, or as the result of an abnormality in the production or composition of tear fluid.

### Levels of Nipbl Expression are Reduced by Only 25-30% in Nipbl⁵⁶⁴/+ Mice

To measure the levels of Nipbl mRNA in Nipbl⁵⁶⁴/+ mice, the inventors developed an RNase protection assay based on hybridization to sequences found in exons 10 and 11. There is no EST evidence supporting alternative splicing of these exons, and in situ hybridization in mouse tissues indicates they are ubiquitously expressed, so it was felt that levels of mRNA containing these exons would provide a good indication of overall Nipbl transcript levels. Because these exons are downstream of the gene trap insertion in the Nipbl⁵⁶⁴/+ allele, and the insertion is designed to terminate transcription, their levels should provide an indication of only wildtype messages.

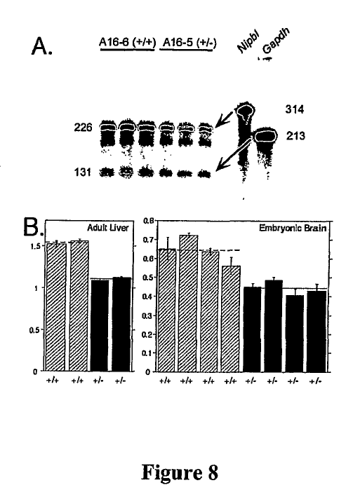

Total RNA was prepared from two tissues: Adult liver, and E18.5 brain, using age-matched littermate controls. Hybridization was carried out simultaneously for Nipbl and the housekeeping gene glyceraldehyde 3-phosphate dehydrogenase (Gadph), so that Nipbl transcript levels could be expressed relative to an internal standard for each sample. Data were averaged for quadruplicate or triplicate samples. The results of several measurements are summarized in FIG. 8: A. Autoradiogram, showing Nipbl and Gapdh probes, and protected fragments of 226 and 131 bases, respectively. RNA samples were prepare from the livers of two female littermates sacrificed at 119 days of age. B. Quantification of Nipbl/Gapdh ratios. Hatched bars represent wildtype animals, filled bars heterozygous mutants. Error bars=standard deviations. Samples from adult liver were from female littermates sacrificed at 73 days of age. Samples from embryonic brain were from 8 animals within a single E17.5 litter. In both cases, data are errors ±s.e.m.

From these and other data it was estimated that mutant Nipbl/Gapdh ratios are 78% of wild type for adult liver and 69% for embryonic brain. Nipbl levels in embryonic brain were ˜70% of those in wildtype animals; in adult liver the values were between 72% and 82%.

The fact that these values are substantially higher than 50% could be explained in either of two ways. One possibility is that the Nipbl⁵⁶⁴/+ allele is “leaky”, i.e. despite the gene trap it makes a certain amount of wild type message. The other possibility is that the Nipbl gene is autoregulatory, such that its expression is upregulated in response to partial loss of its function. Without wishing to be bound by any theory or hypothesis, the inventors favor the second interpretation for two reasons: First, Rollins et al. (Genetics (1999) 152, 577-593) reported that heterozygotes for null alleles of Drosophila Nipped-B also exhibit only a 25% reduction in Nipped-B transcript levels, implying Nipped-B autoregulation in the fly. Second, in a recent study NIPBL transcript levels in human blood cells were assessed by quantitative PCR for an individual with a splice site mutation in NIPBL, and these levels were only 30% lower than that of an unaffected control (Human mutation (2006) 27, 731-735).

### Transcriptional Dysregulation in Nipbl⁵⁶⁴/+ Mice

As described earlier, it has been suggested that the symptoms and pathology of CdLS are caused by transcriptional dysregulation, due to inability to appropriately relieve insulating effects of cohesins in long-range enhancer-promoter communication. Note that, although NIPBL affects transcription in this model, it is not a transcription factor in the usual sense of the word. Transcription factors are proteins that interact directly or indirectly with specific DNA sequences to activate or repress transcription of sets of genes that typically share some functional connection. In contrast, there is no reason to expect that genes whose transcription is altered when NIPBL is impaired are related in any functional way (i.e. that their gene products should participate in common developmental or physiological events). Rather, one should expect such genes to be related only in a structural sense (i.e. they might have enhancers that are particularly distant, or particularly weak, or particularly close to cohesin binding sites). This makes it nearly impossible to predict a priori which genes might be the “targets” of Nipbl loss of function. Accordingly, identification of such targets is best accomplished using whole-genome methods, such as transcriptional profiling by RNA hybridization to whole genome oligonucleotide arrays.

In transcriptional profiling performed by the inventos, RNA derived from the livers of three sex-matched littermate pairs of adult wildtype and Nipbl⁵⁶⁴/+ mice was used. Liver was chosen because it is an organ in which no pathology has been reported in CdLS nor was any noticed in the mice; thus any transcriptional changes the inventors observed would be more likely direct consequences of Nipbl dysfunction, rather than secondary effects of ongoing pathological processes. A “heat map” diagram (FIG. 1) shows those genes that are most substantially and consistently up- or down-regulated in the heterozygous mutant condition. Average changes in expression of such genes varied between 2 and 10 fold. The list of affected genes included members of a wide range of functional classes, including metabolic enzymes, transcription factors, components of signal transduction pathways, and cell-surface receptors. Thus, the data support the hypothesis that partial loss of Nipbl function results in significant transcriptional dysregulation of many genes.

Thus, specific embodiments and applications of compositions and methods relating to Cornelia De Lange syndrome have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A method of diagnosing a person as having Cornelia de Lange syndrome, comprising:
identifying a surrogate marker, wherein the surrogate marker is a gene that is over-expressed or under-expressed as a function of a NIPBL+/− genotype;
measuring expression of the surrogate marker to obtain a test result; and
diagnosing the person as having the Cornelia de Lange syndrome using the test result.

2. The method of claim 1 wherein the NIPBL+/− genotype is caused by knockout or knockdown of at least one allele of the NIPBL gene.

3. The method of claim 1 wherein the NIPBL+/− genotype is caused by a mutation in at least one allele of the NIPBL gene, wherein the mutation is a mutation known to be associated with Cornelia de Lange syndrome.

4. The method of claim 1 wherein the surrogate marker is a gene that is over-expressed or under-expressed at least 1.5 times as compared to expression under NIPBL+/+ genotype.

5. The method of claim 1 wherein the surrogate marker is a gene that is over-expressed or under-expressed at least 2.0 times as compared to expression under NIPBL+/+ genotype.

6. The method of claim 1 wherein the step of measuring comprises multi-gene analysis.

7. The method of claim 4 wherein multi-gene analysis comprises quantitative PCR or gene chip analysis.

8. The method of claim 1 wherein the step of measuring comprises quantitative analysis of a gene product of the gene that is over-expressed or under-expressed.

9. The method of claim 1 further comprising a step of identifying a second surrogate marker, wherein the second surrogate marker is a second gene that is over-expressed or under-expressed as a function of a NIPBL+/− genotype, and further comprising measuring expression of the second surrogate marker to obtain the test result.

10. The method of claim 1 wherein the surrogate marker is selected from the group of sequences consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, and SEQ ID NO 38.

11. A kit comprising:
a genetically modified animal comprising a cell with NIPBL+/− genotype; and
an instruction associated with the animal to use the animal as a model for Cornelia de Lange syndrome.

12. The kit of claim 11 wherein the animal is a transgenic mouse in which one allele of the NIPBL gene is inactivated or eliminated.

13. The kit of claim 11 wherein the animal is a transgenic mouse in which at least one NIPBL allele has a mutation that is associated with Cornelia de Lange syndrome.

14. The kit of claim 11 wherein the cell with NIPBL+/− genotype is a cell selected from the group consisting of a neural cell, a hepatic cell, and a dermal cell.

15. The kit of claim 11 wherein the instruction comprises an information to measure expression of a surrogate marker, wherein the surrogate marker is a gene that is over-expressed or under-expressed as a function of a NIPBL+/− genotype.

16. The kit of claim 15 wherein the instructions informs that measurement of the expression of the surrogate marker is performed after administration of a potential therapeutic agent.

17. A method of identifying a diagnostic marker or therapeutic target for treatment of Cornelia de Lange syndrome, comprising:
providing an animal model according to claim 11; and
identifying a surrogate marker, wherein the surrogate marker is a gene that is over-expressed or under-expressed as a function of a NIPBL+/− genotype;
optionally administering to the animal a potential therapeutic agent; and
optionally monitoring change in expression of the surrogate marker after administration of the potential therapeutic agent.

18. The method of claim 17 wherein the potential therapeutic agent increases expression of NIPBL or is a recombinant NIPBL gene or protein.

19. The method of claim 17 wherein the potential therapeutic agent is administered and wherein change in expression of the surrogate marker after administration of the potential therapeutic agent is measured.

20. The method of claim 17 wherein the surrogate marker is selected from the group of sequences consisting of SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 19, SEQ ID NO 20, SEQ ID NO 21, SEQ ID NO 22, SEQ ID NO 23, SEQ ID NO 24, SEQ ID NO 25, SEQ ID NO 26, SEQ ID NO 27, SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, and SEQ ID NO 38.


SEQUENCE LISTING (38 sequences)

SEQ ID NO: 1 - DNA, 2068 bp, Homo sapiens
  misc_feature (1)..(2068): upregulated in mutant
gcagtctccagcctgagccatgggccgccgagccctcctgctcctgcttctgtcttttctggcgccctgggccaccatagccctccggccggccttaagggccctcggcagcctacacttgccaaccaaccccacatccctcccggctgtagccaagaactattcggttctctacttccaacagaaggttgatcattttggatttaatactgtgaaaacttttaatcagcggtacctagtagctgataaatactggaagaaaaatggtggatcaatacttttctacactggtaatgaaggggacattatctggttttgtaataacacggggttcatgtgggatgtggctgaggaactgaaagctatgttggtgtttgctgaacatcgatactatggagagtctctcccctttggtgacaactcattcaaggattccagacacttgaatttcctgacatcagaacaagctctggctgattttgcagagttaatcaaacacttgaaaagaacaatcccaggagctgaaaatcaacctgtcattgccataggaggctcctatggtggcatgcttgccgcctggtttaggatgaaatatcctcatatggtagttggagctcttgcagcttctgcccctatctggcagtttgaggatttagtaccttgtggtgtatttatgaagatcgtaactacagattttaggaaaagcggtccacattgttcagagagcatccacaggtcctgggatgccattaatcgactctcaaatactggcagtggtttgcagtggcttactggagcccttcacttatgcagcccattaacttctcaggacatccaacatttgaaagactggatctctgaaacctgggtgaatctggcaatggtggactatccttatgcctctaactttttacagcctttgcctgcttggcctatcaaggtagtgtgccagtatttgaaaaatcccaatgtatctgattcactgctgctgcagaatattttccaagctctgaatgtatattacaattattcgggccaggtgaaatgcctgaatatttcagagacagcaactagcagtctgggaacactgggttggagctatcaggcctgcacagaagtagtcatgcccttttgtactaatggtgtcgatgacatgtttgaacctcactcatggaacttaaaggaactttctgatgactgttttcaacagtggggtgtgagaccaaggccctcctggatcactactatgtatggaggcaaaaacattagttcacacacaaacattgttttcagcaatggtgaactagacccctggtcaggaggtggagtaactaaggatatcacagacactctggttgcagtcaccatctcagagggggcccaccacttagatctccgcaccaagaatgccttggatcctatgtctgtgctgttagcccgctccttggaagttagacatatgaagaattggatcagagatttctatgacagtgcgggaaagcagcactgagaaacttttgattgttttcaatttcttcttttatgttcacaccaccacattcccattcactttgattttctacatgtaattaccttcttttgtttatcattagatttgatggggccaaagttgagatagaatagagggtgatgacggtaagagcaagtgtcccatgaatgtgatttcctgggttctcactgtctttgcaccacgtctaggaagaatcttcttgatagctctcccacaccatcagtggccctcataactggagtagagttcctggttgcttttcataagagggagagttactttctttgtatctctgcaagcagagatttctctttggttttgaggttgaagtgtctttggcccatttgtaagtccccatccctaccctacacaaagtaaaagcagaagatagataaaaaaatgatgtaattgcagctggtaggatgtctggtgccaatccaggaagtgagagccatttcttttgtaccggatttaatgactttgaactgtgctgtaaataaataatacagctggaccttaaaaaaaaaaaaaaaaaaa

SEQ ID NO: 2 - DNA, 1365 bp, Homo sapiens
  misc_feature (1)..(1365): upregulated in mutant
atgtctccaccaccgctgctgcaacccctgctgctgctgctgcctctgctgaatgtggagccttccggggccacactgatccgcatccctcttcatcgagtccaacctggacgcaggatcctgaacctactgaggggatggagagaaccagcagagctccccaagttgggggccccatcccctggggacaagcccatcttcgtacctctctcgaactacagggatgtgcagtattttggggaaattgggctgggaacgcctccacaaaacttcactgttgcctttgacactggctcctccaatctctgggtcccgtccaggagatgccacttcttcagtgtgccctgctggttacaccaccgatttgatcccaaagcctctagctccttccaggccaatgggaccaagtttgccattcaatatggaactgggcgggtagatggaatcctgagcgaggacaagctgactattggtggaatcaagggtgcatcagtgattttcggggaggctctctgggagcccagcctggtcttcgcttttgcccattttgatgggatattgggcctcggttttcccattctgtctgtggaaggagttcggcccccgatggatgtactggtggagcaggggctattggataagcctgtcttctccttttacctcaacagggaccctgaagagcctgatggaggagagctggtcctggggggctcggacccggcacactacatcccacccctcaccttcgtgccagtcacggtccctgcctactggcagatccacatggagcgtgtgaaggtgggcccagggctgactctctgtgccaagggctgtgctgccatcctggatacgggcacgtccctcatcacaggacccactgaggagatccgggccctgcatgcagccattgggggaatccccttgctggctggggagtacatcatcctgtgctcggaaatcccaaagctccccgcagtctccttccttcttgggggggtctggtttaacctcacggcccatgattacgtcatccagactactcgaaatggcgtccgcctctgcttgtccggtttccaggccctggatgtccctccgcctgcagggcccttctggatcctcggtgacgtcttcttggggacgtatgtggccgtcttcgaccgcggggacatgaagagcagcgcccgggtgggcctggcgcgcgctcgcactcgcggagcggacctcggatggggagagactgcgcaggcgcagttccccgggtgacgcccaagtgaagcgcatgcgcagcgggtggtcgcggaggtcctgctacccagtaaaaatccactatttccattgagaaaaaacaaaaaaaaaaaaaaaaaa

SEQ ID NO: 3 - DNA, 828 bp, Homo sapiens
  misc_feature: upregulated in mutant
tcgagtttggtctccaataaaaaggctatctttattaggaagggcttgagttactagggaagagcttcgcgcgcctacactaggcgctgaaatgggatgctggggcttggtggctccggcgggagcagctggtagggctagggctccctggccccccttgaaggggttgggctgcgtgggtgggggctgtgcggggctccgggggccacactcacgccctgtgtcgcccgcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaaccc

SEQ ID NO: 4 - DNA, 1698 bp, Homo sapiens
  misc_feature (1)..(1698): upregulated in mutant
ggttactcatcctgggctcaggtaagagggcccgagctcggaggcggcacacccaggggggacgccaagggagcaggacggagccatggaccccgccaggaaagcaggtgcccaggccatgatctggactgcaggctggctgctgctgctgctgcttcgcggaggagcgcaggccctggagtgctacagctgcgtgcagaaagcagatgacggatgctccccgaacaagatgaagacagtgaagtgcgcgccgggcgtggacgtctgcaccgaggccgtgggggcggtggagaccatccacggacaattctcgctggcagtgcsgggttgcggttcgggactccccggcaagaatgaccgcggcctggatcttcacgggcttctggcgttcatccagctgcagcaatgcgctcaggatcgctgcaacgccaagctcaacctcacctcgcgggcgctcgacccggcaggtaatgagagtgcatacccgcccaacggcgtggagtgctacagctgtgtgggcctgagccgggaggcgtgccagggtacatcgccgccggtcgtgagctgctacaacgccagcgatcatgtctacaagggctgcttcgacggcaacgtcaccttgacggcagctaatgtgactgtgtccttgcctgtccggggctgtgtccaggatgaattctgcactcgggatggagtaacaggcccagggttcacgctcagtggctcctgttgccaggggtcccgctgtaactctgacctccgcaacaagacctacttctcccctcgaatcccaccccttgtccggctgccccctccagagcccacgactgtggcctcaaccacatctgtcaccacttctacctcggccccagtgagacccacatccaccaccaaacccatgccagcgccaaccagtcagactccgagacagggagtagaacacgaggcctcccgggatgaggagcccaggttgactggaggcgccgctggccaccaggaccgcagcaattcagggcagtatcctgcaaaaggggggccccagcagccccataataaaggctgtgtggctcccacagctggattggcagcccttctgttggccgtggctgctggtgtcctactgtgagcttctccacctggaaatttccctctcacctacttctctggccctgggtacccctcttctcatcacttcctgttcccaccactggactgggctggcccagcccctgtttttccaacattccccagtatccccagcttctgctgcgctggtttgcggctttgggaaataaaataccgttgtatatattctggcaggggtgttctagctttttgaggacagctcctgtatccttctcatccttgtctctccgcttgtcctcttgtgatgttaggacagagtgagagaagtcagctgtcacggggaaggtgagagagaggatgctaagcttcctactcactttctcctagccagcctggactttggagcgtggggtgggtgggacaatggctccccactctaagcactgcctcccctactccccgcatctttggggaatcggttccccatatgtcttccttactagactgtgagctcctcgagggcagggaccgtgccttatgtctgtgtgtgatcagtttctggcacataaatgcctcaataaagatttaattactttgtatagtgaaaaaaaa

SEQ ID NO: 5 - DNA, 2802 bp, Homo sapiens
  misc_feature (1)..(2802): upregulated in mutant
atgacggcgacccctgagagcctcttccccactggggacgaactggactccagccagctccagatggagtccgatgaggtggacaccctgaaggagggagaggacccagccgaccggatgcacccgtttctggccatctatgagcttcagtctctgaaagtgcaccccttggtgttcgcacctggggtccctgtcacagcccaggtggtgggcaccgaaagatataccagcggatccaaggtgggaacctgcactctgtattctgtccgcttgactcacggcgacttttcctggacaaccaagaagaaataccgtcattttcaggagctgcatcgggacctcctgagacacaaagtcttgatgagtctgctccccctggctcgatttgccgttgcctattctccagcccgagatgcaggcaacagagagatgccctctctaccccgggcaggtcctgagggctccaccagacatgcagccagcaaacagaaatacctggagaattacctcaaccgtctcttgaccatgtctttctatcgcaactaccatgccatgacagagttcctggaagtcagtcagctgtcctttatcccggacttgggccgcaaaggactggaggggatgatccggaagcgctcaggtggccaccgtgttcctggcctcacctgctgtggccgagaccaagtttgttatcgctggtccaagaggtggctggtggtgaaggactccttcctgctgtacatgtgcctcgagacaggtgccatctcatttgttcagctctttgaccctggctttgaggtgcaagtggggaaaaggagcacggaggcacggcacggcgtgcggatcgatacctcccacaggtccttgattctcaagtgcagcagctaccggcaggcacggtggtgggcccaagagatcactgagctggcgcagggcccaggcagagacttcctacagctgcaccggcatgacagctacgccccaccccggcctgggaccttggcccggtggtttgtgaatggggcaggttactttgctgctgtggcagatgccatccttcgagctcaagaggagattttcatcacagactggtggttgagtcctgaggtttacctgaagcgtccggcccattcagatgactggagactggacatcatgctcaagaggaaggcggaggaaggtgtccgtgtgtctattctgctgtttaaagaagtggaattggccttgggcatcaacagtggctatagcaagagggcgctgatgctgctgcaccccaacataaaggtgatgcgtcacccagaccaagtgacgttgtgggcccatcatgagaagctcctggtggtggaccaagtggtagcattcctggggggactggaccttgcctatggccgctgggatgacctgcactaccgactgactgaccttggagactcctctgaatcagctgcctcccagcctcccaccccgcgcccagactcaccagccaccccagacctctctcacaaccaattcttctggctgggcaaggactacagcaatcttatcaccaaggactgggtgcagctggaccggcctttcgaagatttcattgacagggagacgacccctcggatgccatggcgggacgttggggtggtcgtccatggcctaccggcccgggaccttgcccggcacttcatccagcgctggaacttcaccaagaccaccaaggccaagtacaagactcccatatacccctacctgcttcccaagtctaccagcacggccaatcagctccccttcacacttccaggagggcagtgcaccaccgtacaggtcttgcgatcagtggaccgctggtcagcagggactctggagaactccatcctcaatgcctacctgcacaccatcagggagagccagcacttcctctacattgagaatcagttcttcattagctgctcagatgggcggacggttctgaacaaggtgggcgatgagattgtggacagaatcctgaaggcccacaaacaggggtggtgttaccgagtctacgtgcttttgcccttactccctggcttcgagggtgacatctccacgggcggtggcaactccatccaggccattctgcactttacttacaggaccctgtgtcgtggggagtattcaatcctgcatcgccttaaagcagccatggggacagcatggcgggactatatttccatctgcgggcttcgtacacacggagagctgggcgggcaccccgtctcggagctcatctacatccacagcaaggtgctcatcgcagatgaccggacagtcatcattggttctgcaaacatcaatgaccggagcttgctggggaagcgggacagtgagctggccgtgctgatcgaggacacagagacggaaccatccctcatagatggggcagagtatcaggcgggcaggtttgccttgagtctgcggaagcactgcttcggtgtgattcttggagcaaatacccggccagacttggatctccgagaccccatctgtgatgacttcttccagttgtggcaagacatggctgagagcaacgccaatatctatgagcagatcttccgctgcctgccatccaatgccacgcgttccctgcggactctccgggagtacgtggccgtggagcccttggccacggtcagtccccccttggctcggtctgagctcacccaggtccagggccacctggtccacttccccctcaagttcctagaggatgagtctttgctgcccccgctgggtagcaaggagggcatgatccccctagaagtgtggacatag

SEQ ID NO: 6 - DNA, 5820 bp, Homo sapiens
  misc_feature (1)..(5820): upregulated in mutant
atgaccgatgcccagatggctgactttggggcagcggcccagtacctccgcaagtcagagaaggagcgtctagaggcccagacccggccctttgacattcgcactgagtgcttcgtgcccgatgacaaggaagagtttgtcaaagccaagattttgtcccgggagggaggcaaggtcattgctgaaaccgagaatgggaagacggtgactgtgaaggaggaccaggtgttgcagcagaacccacccaagttcgacaagattgaggacatggccatgctgaccttcctgcacgagcccgcggtgcttttcaacctcaaggagcgctacgcggcctggatgatatatacctactcgggcctcttctgtgtcactgtcaacccctacaagtggctgccggtgtacaatgccgaggtggtggccgcctaccggggcaagaagaggagtgaggccccgccccacatcttctccatctccgacaacgcctatcagtacatgctgacagatcgggagaaccagtccatcctcatcacgggagaatccggggcggggaagactgtgaacaccaagcgtgtcatccagtactttgccagcattgcagccataggtgaccgtggcaagaaggacaatgccaatgcgaacaagggcaccctggaggaccagatcatccaggccaaccccgctctggaggccttcggcaatgccaagactgtccggaacgacaactcctcccgctttgggaaattcattaggatccactttggggccactggaaagctggcttctgcagacatagagacctacctgctggagaagtcccgggtgatcttccagctgaaagctgagagaaactaccacatcttctaccagattctgtccaacaagaagccggagttgctggacatgctgctggtcaccaacaatccctacgactacgccttcgtgtctcagggagaggtgtccgtggcctccattgatgactccgaggagctcatggccaccgatagtgcctttgacgtgctgggcttcacttcagaggagaaagctggcgtctacaagctgacgggagccatcatgcactacgggaacatgaagttcaagcagaagcagcgggaggagcaggcggagccagacggcaccgaagatgctgacaagtctgcctacctcatggggctgaactcagctgacctgctcaaggggctgtgccaccctcgggtgaaagtgggcaacgagtatgtcaccaaggggcagagcgtgcagcaggtttactactccatcggggctctggccaaggcagtgtatgagaagatgttcaactggatggtgacgcgcatcaacgccaccctggagaccaagcagccacgccagtacttcataggagtcctggacatcgctggcttcgagatcttcgacttcaacagctttgagcagctctgcatcaacttcaccaacgagaagctgcagcagttcttcaaccaccacatgttcgtgctggagcaggaggagtacaagaaggagggcattgagtggacattcattgactttggcatggacctgcaggcctgcattgacctcatcgagaagcccatgggcatcatgtccatcctggaggaggagtgcatgttccccaaggccactgacatgaccttcaaggccaagctgtacgacaaccacctgggcaagtccaacaatttccagaagccacgcaacatcaaggggaagcctgaagcccacttctccctgatccactacgccggcactgtggactacaacatcctgggctggctggaaaaaaacaaggatcctctcaacgagactgtcgtgggcttgtatcagaagtcctccctcaagctcatggccactctcttctcctcctacgcaactgccgatactggggacagtggtaaaagcaaaggaggcaagaaaaagggctcatccttccagacggtgtcggctctccaccgggaaaatctcaacaagctaatgaccaacctgaggaccacccatcctcactttgtgcgttgtatcatccccaatgagcggaaggctccaggggtgatggacaaccccctggtcatgcaccagctgcgctgcaatggcgtgctggagggcatccgcatctgcaggaagggcttccccaaccgcatcctctacggggacttccggcagaggtatcgcatcctgaacccagtggccatccctgagggacagttcattgatagcaggaagggggcagagaagctgctcagctctctggacattgatcacaaccagtacaagtttggccacaccaaggtgttcttcaaggccgggctgctggggctgctggaggaaatgagggacgagaggctgagccgcatcatcacgcgtatccaggcccaagcccggggccagctcatgcgcattgagttcaagaagatagtggaacgcagggatgccctgctggtaatccagtggaacattcgggccttcatgggggtcaagaattggccctggatgaagctctacttcaagatcaagccgctgctgaagagcgcagagacggagaaggagatggccaccatgaaggaagagttcgggcgcatcaaagagacgctggagaagtccgaggctcgccgcaaggagctggaggagaagatggtgtccctgctgcaggagaagaatgacctgcagctccaagtgcaggcggaacaagacaacctcaatgatgctgaggagcgctgcgaccagctgatcaaaaacaagattcagctggaggccaaagtaaaggagatgaatgagaggctggaggatgaggaggagatgaacgcggagctcactgccaagaagcgcaagctggaagatgagtgctcagagctcaagaaggacattgatgacctggagctgacactggccaaggtggagaaggagaagcatgcaacagagaacaaggtgaagaacctaacagaggagatggctgggctggatgaaatcatcgctaagctgaccaaggagaagaaagctctacaagaggcccatcagcaggccctggatgaccttcaggccgaagaagacaaggtcaacaccctgtccaagtctaaggtcaagctggagcagcaggtggatgatctggagggatccctagagcaagagaagaaggtgcgcatggacctggagcgagcaaagcggaagctggagggtgacttgaagctgacccaggagagcatcatggacctggaaaatgataaactgcagctggaagaaaagcttaagaagaaggagtttgacattaatcagcagaacagtaagattgaggatgagcaggtgctggcccttcaactacagaagaaactgaaggaaaaccaggcacgcatcgaggagctggaggaggagctggaggccgagcgcaccgccagggctaaggtggagaagctgcgctcagacctgtctcgggagctggaggagatcagcgagcggctggaagaggccggcggggcaacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaaacctggagaaagtgtctcggacgctggaggaccaggccaatgagtaccgcgtgaagctagaagaggcccaacgctccctcaatgatttcaccacccagcgagccaagctgcagaccgagaatggagagttgtcccggcagctagaggaaaaggaggcgctaatctcgcagcttacccgtgggaagctctcttatacccagcaaatggaggacctcaaaaggcagctggaggaggagggcaaggcgaagaacgccctggcccatgcactgcagtcggcccggcatgactgcgacctgctgcgggagcagtacgaggaggagacagaggccaaggccgagctgcagcgcgtcctgtccaaggccaactcggaggtggcccagtgtaggaccaagtatgagacggacgccattcagcggactgaggagctcgaagaggccaaaaagaagctggcccagcggctgcaggatgccgaggaggccgtggaggctgttaatgccaagtgctcctcactggagaagaccaagcaccggctacagaatgagatagaggacttgatggtggacgtagagcgctccaatgctgctgctgcagccctggacaagaagcagagaaactttgacaagatcctggccgagtggaagcagaagtatgaggagtcgcagtctgagctggagtcctcacagaaggaggctcgctccctcagcacagagctcttcaagctcaagaacgcctacgaggagtccctggagcacctagagaccttcaagcgggagaacaagaaccttcaggaggaaatctcggaccttactgagcagctaggagaaggaggaaagaatgtgcatgagctggagaaggtccgcaaacagctggaggtggagaagctggagctgcagtcagccctggaggaggcagaggcctccctggagcacgaggagggcaagatcctccgggcccagctagagttcaaccagatcaaggcagagatcgagcggaagctggcagagaaggacgaggagatggaacaggccaagcgcaaccaccagcgggtggtggactcgctgcagacctccctggatgcagagacacgcagccgcaacgaggtcctgagggtgaagaagaagatggaaggagacctcaatgagatggagatccagctcagccacgccaaccgcatggctgccgaggcccagaagcaagtcaagagcctccagagcttgctgaaggacacccagatccagctggacgatgcggtccgtgccaacgacgacctgaaggagaacatcgccatcgtggagcggcgcaacaacctgctgcaggctgagctggaggagctgcgtgccgtggtggagcagacagagcggtcccggaagctggcggacagggagctgattgagaccagcgagcgggtgcagctgctgcattcccagaacaccagcctcatcaaccagaagaagaagatggatgctgacctgtcccagctccagtcggaagtggaggaggcagtgcaggagtgcagaaacgccgaggagaaggccaagaaggccatcacgcatgccgccatgatggcagaggagctgaagaaggagcaggacaccagcgcccacctggagcgcatgaagaagaacatggagcagaccattaaggacctgcagcaccggctggacgaggccgagcagatcgccctcaagggcggcaagaagcagctgcagaagctggaagcgcgggtgcgggagctggagggtgagctggaggccgagcagaagcgcaacgcagagtcggtgaagggcatgaggaagagcgagcggcgcatcaaggagctcacctaccagacagaggaagacaaaaagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtga

SEQ ID NO: 7 - DNA, 1356 bp, Homo sapiens
  misc_feature (1)..(1356): upregulated in mutant
gtatatataacgtgatgagcgtacgggtgcggagacgcaccggagcgctcgcccagccgccgyctccaagcccctgaggtttccggggaccacaatgaacaagttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgataa

SEQ ID NO: 8 - DNA, 1441 bp, Homo sapiens
  misc_feature (1)..(1441): upregulated in mutant
ccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgacccccag

SEQ ID NO: 9 - DNA, 1369 bp, Homo sapiens
  misc_feature (1)..(1369): upregulated in mutant
cctttctggaagctgcagggctctccatccaggatccagaagcattgaaggggaccagccgctgaagggattctcagtcccatctgactccccatgaggctcctggctttcctgagtctgctggccttggtgctgcaggagacagggacagcttctctcccaaggaaggagaggaagaggagagaggagcagatgcccagggaaggcgattcctttgaagttctgcctctgcggaatgatgtcctgaacccagacaactatggtgaagtcattgacctgagcaactatgaggagctcacagattatggggaccaactccccgaggttaaggtgactagcctcgctcctgcaaccagcatcagtcccgccaagagcactacggctccagggacaccctcgtcaaaccccacgatgaccagacctactacagcagggctgctactgagttcccagcccaaccatggtctgcccacctgcctggtctgcgtgtgcctcggttcctctgtgtattgcgatgacattgacctagaggacattcctcctcttcctcggaggactgcctacctgtatgcacgcttcaaccgcatcagccgtatcagggccgaagacttcaaagggctgacaaagttgaagaggattgacctctccaacaacctcatttcctccatcgataatgatgccttccgcctgctacatgccctccaggacctcatcctcccagagaaccagttggaagctctgcccgtgctgcccagtggcattgagttcctggatgtccgcctaaatcggctccagagctcggggatacagcctgcagccttcagggcaatggagaagctgcagttcctttacctgtcagacaacctgctggattctatcccggggcctttgcccctgagcctgcgctctgtacacctgcagaataacctgatagagaccatgcagagagacgtcttctgtgaccccgaggagcacaaacacacccgcaggcagctggaagacatccgcctggatggcaaccccatcaacctcagcctcttccccagcgcctacttctgcctgcctcggctccccatcggccgcttcacgtagctcggagcccttccactcctcccaggtcatctcttggaccagcgggcatcacattctccagcagccgccatctcacacgcctccctcctgtggccgccggcagcatggacaaaggtctccatgcagggggaggaggcctgcttctttccccacagctctcacgtctcccttctccctgcgggtgacaaagaagcccaaggaccacctccttcctgcctcattgtaataaaattccccacactgagaaaaaaaaaaaaaaaaaaaaaaaaaaaaaaaa

SEQ ID NO: 10 - DNA, 1817 bp, Homo sapiens
  misc_feature (1)..(1817): upregulated in mutant
cgggatctgtgtgaagtggactaaggattaagtaggatgtcaactgagacagaacttcaagtagctgtgaaaaccagcgccaagaaagactccagaaagaaaggtcaggatcgcagtgaagccactttgataaagaggtttaaaggtgaaggggtccggtacaaagccaaattgatcgggattgatgaagtttccgcagctcggggagacaagttatgtcaagattccatgatgaaactcaagggcgttgttgctggcgctcgttccaaaggagaacacaaacagaaaatctttttaaccatctcctttggaggaatcaaaatctttgatgagaagacaggggcccttcagcatcatcatgctgttcatgaaatatcctacattgcaaaggacattacagatcaccgggcctttggatatgtttgtgggaaggaagggaatcacagatttgtggccataaaaacagcccaggcggctgaacctgttattctggacttgagagatctctttcaactcatttatgaattgaagcaaagagaagaattagaaaaaaaggcacaaaaggataagcagtgtgaacaagctgtgtaccaggttcccaccagccaaaagaaggaaggtgtttatgatgtgccaaaaagtcaacctgtaagtaatggctattcgtttgaggattttgaagaacggtttgctgcagccaccccgaacagaaacctgcccacagactttgatgagatttttgaggcaacgaaggctgtgacccaattagaactttttggggacatgtccacaccccctgatataacctctccccccactcctgcaactccaggtgatgcctttatcccatcttcatctcagacccttccagcgagtgcagatgtgtttagttctgtacctttcggcactgctgctgtaccctcaggttacgttgcaatgggcgctgtcctcccgtccttctggggtcagcagcccctcgtccaacagcagatggtcatgggtgcccagcctccagtcgctcaggtgatgccgggggctcagcccatcgcatggggccagccgggtctctttcctgccactcagcagccctggccaactgtggccgggcagtttccgccagccgccttcatgcccacacaaactgttatgcctttgccagctgccatgttccaaggtcccctcaccccccttgccaccgtcccaggcacgagtgactccaccaggtcaagtccacagaccgacaagcccaggcagaaaatgggcaaagaaacgtttaaggatttccagatggcccagcctccgcccgtgccctcccgcaaacccgaccagccctccctcacctgtacctcagaggccttctccagttacttcaacaaagtcggggtggcacaggatacagacgactgtgatgactttgacatctcccagttgaatttgacccctgtgacttctaccacaccatcgaccaactcacctccaaccccagcccctagacagagctctccatccaaatcatctgcatcccatgccagtgatcctaccacagatgacatctttgaagagggctttgaaagtcccagcaaaagcgaagagcaagaagctcctgatggatcacaggcctcatccaacagtgatccatttggtgagcccagtggggagcccagtggtgataatataagtccacaggccggtagctagatagcgcaggtctgggagccagagcctctgtacgcgcagatcaacagacctaagaaatagcatcgatgcgagctcgtggtgggtgctcaagactggcatggacatcagcatcacgacag

SEQ ID NO: 11 - DNA, 6099 bp, Homo sapiens
  misc_feature (1)..(6099): upregulated in mutant
ccggcggccgagcgttctgagtcacccgggactggaggtaggaacccagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagtgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcagtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctctagccaggagtctgtgctcttttggtggggagcacttgcgtcctgcagaggaccacagtgggtggcacctcctgagaaggcgaggagagtggttgttgccaactaagccctcgaaccaaggcagcctccagagccagcctgggactcccagtgaacttacacttggagcccgtgcagtacaggcaaaacacgcaagggcatcaggcactggtggcatcgtagaagagatgtggcaaagtgctgtacccttccacctcctagaggtgggcagctgggccccacctacttgtgactgaaggggcacaccactgccctgcctgcccacttagccgtccatggcaccagccccctggatgggcattgggctgacacctaccatgctgctttttggcacagttgtctattctgagccttgagagaaaaagtgccccttaagggttgaaggcagtctgaacccttgtgcttggtggggctcgtggccttccccttttgcctggctgtggaggcctgatgctgccccgttccctgtcagaggctaagatgagatttgccagcacaggggccccagatctgcctgggcctgtgcagcagcccagcttcctggtgtatttttcaggtaggcccttgtcctgccagctgccttcctcatcccctcgtcctgtcccagaggttatctgcctggcctggctccccacgagtcacctgcaagccccagggcctgggggcagtgactggcaggtgcagatgggctgtttcgtgtagtggaagagcagcctgatggccaagggggtggacgcaattgtgggatgtcctctttactcccttcctggcctcactggctggggcagaggggcagccgctaggagagactgaaagcagcagctaggactgaggagtgggttttattgtccttcagagctcttcaagctgtcccctctgtcatcactccctggatgtgtggggcatggttccttccctgggaaggctaagttcagttctgttttttattctatgagaacaagtcacagctgcagctgggccccatgctctgccccaagcccccaaccccgcggtgctccggcggcttcctgtccactctcggggcccttggggcctggcttgctccagggtcttgggctactggcagctcctctccttgggctcctggctgccaggcgttggtgccacttcttaaaggcctggaaccagggaggagaggaaatgctattgttgtgggctttctccggggtctgtgctgtgcctgctagagcaacccctgtacccagctccttttgtccccagggcccctccctctgccccaagcagccagccagtcttgcctaggccaaatgcacaagctcagaatagatctgatggtgagctgggaagctgtactcagagcagagcaaatgagggagggggcgctcaggacccaggccctccatgggctagtgtgagtggcagccatgcctcatgccacaccttcttcgcaaactgatggaccgggtgggcctggcctgagctggggccacaaatcaaagcaagggctccagtatccagcctgtgtgttctgtaatggaactgaccccctcccctgaaaacgaaggggccccggggctggcaagcagggaaagctccacggtgcgcggctgtggcacagacttctggaaggctggctgagtggaatgcagggaagagggcagtacctgggaaaggacccacccatcttcctgctgctgtaactgctgagccactcgcagtcgcaggatccgctgccaccacgtctgccaggcccatctcaggtgccactccctgagctttggggacagttggcagagaaggcctcttgtgctcacgctcccccgcagtccccagcccttctgcctttctcccccgacactgctgcaccagagtgaaagggctatggcaagggggtgtcatctgaggagtattaagaatgcagattcctgggcctgtcccccaaggttttggagtcagtaggtccaagggccatacttttgagaggggtttgggttaagtatgaggtgaaatgggagatggtcagtgtggagaggggtgcacccactcaccagggtccgcaccagctgctctgccccttgggcatccacccagtgctgccatgccactgccaggcacctggcctgctgggaaccccgcagcccgtgaagcagtgcctcgaggcaccggcgctgcaggtacttcctcctgatggccaagagcatcgtgacccttcagggccagaaggagggcagagccatgggcctgggcctgcttttccaggatcctgcaggaacgagcactggccagagagggcccagctgtagccatggctcaggcaagcccctcagcccttgcccccatccctcggacccaccaaactgcacacacagctcctcttaccgtagcctccgtttatgggccttgctttgggctttggaggctctgggctcagggctggagtgcgctcttggtccctggtccctcgtccacaggggcaggcctgggacccagctactctgtccaggccactgtggccagagctggaaggcagggcagagggaatgttccctgcaccctggaaaggggagttgagtcacaagagtttaaggtgggtccaggaaggcagctgctcttagtgcccgcttaggagttgagtacagtgaggagggtggaggaaggtgctgagcttagccttgtgccctgcccccatctccccaggcctccagcctctcacggctgcctgccgcccaaagagaaatcacaggggcggggcaggaatgcaaagtgttttctcagaacagctgaaacattccgaagagggaatggatggggagaatggtcaatacacataagaccgtgtcccaaggagctgatttccaggcccctgaggactggagaccgcttcacccctgcacttcagacaccgtttgtcccccggggcaaggtctccttactctgagcccaggccgttccccttggcttcctccgtccacccaggctgcactgcagtgatggcgcgggaggcaccagctctgtggcctgtgtccagcagctgtgggtctgaaggaatagccagagaggagcacctgaaccccatgggcttggacttcctggggccccgctgggatttcttcgctgctctagctggcaggacacatcccggcctcttccacccattcccccatgtggctgaagacattccaacaatggggtgggcccataatagttagccctcagtcagttcccggagcacagccctgggagggggctatttctctccccactgaaaacatttcaaagctgagttacttgtctgaggcctcatccctcggaagccgtctgactccagagtctgagcccccggctagtaccctatagagagggggctctccaaaggggctgctggggcatgtgtgcctgtggcagaaaagaggagaccctggaattcagcaccctgggtgccattcccagcgtttagtttctagaggcctcagtttctccatcagcttatgggatccttgtctttactgacaagaatggaatagaaatgtaaaagtactctgaaaagcaattgccctgtaacttatctagaaagaaaagaccctgagactccagaatctgctgttgccatagccccatatgtgtgaattctgcaactagccaaggctagttcctttcaattccatttaaaaaacaaaaaccagcaggtgtggtggctcatggcgtaatgggcctgcccaatgctttgggaggccaaggcagatagatcgcttgagcccaggagtttgagacaagccctggcaacatagtgagatcccatttctacaaaaaaaaaaaaaaaaaaa

SEQ ID NO: 12 - DNA, 1693 bp, Homo sapiens
  misc_feature (1)..(1693): upregulated in mutant
cacagtcctcggcccaggccaagcaagcttctatctgcacctgctctcaatcctgctctcaccatgagcctccgcctgcagagctcctctgccagctatggaggtggtttcgggggtggctcttgccagctgggaggaggccgtggtgtctctacctgttcaactcggtttgtgtctgggggatcagctgggggctatggaggcggcgtgagctgtggttttggtggaggggctggtagtggctttggaggtggctatggaggtggccttggaggtggctatggaggtggccttggaggtggctttggtgggggttttgctggtggctttgttgactttggtgcttgtgatggcggcctcctcactggcaatgagaagatcaccatgcagaacctcaacgaccgcctggcttcctacctggagaaggtgcgcgccctggaggaggccaacgctgacctggaggtgaagatccgtgactggcacctgaagcagagcccagctagccctgagcgggactacagcccctactacaagaccattgaagagctccgggacaagatcctgaccgccaccattgaaaacaaccgggtcatcctggagattgacaatgccaggctggctgtggacgacttcaggctcaagtatgagaatgagctggccctgcgccagagcgtggaggccgacatcaacggcctgcgccgggtgctggatgagctcactctgtctaagactgacctggagatgcagatcgagagcctgaatgaagagctagcctacatgaagaagaaccatgaagaggagatgaaggaatttagcaaccaggtggtcggccaggtcaacgtggagatggatgccaccccaggcattgacctgacccgcgtgctggcagagatgagggagcagtacgaggccatggcagagaggaaccgccgggatgctgaggaatggttccacgccaagagtgcagagctgaacaaggaggtgtctaccaacactgccatgattcagaccagcaagacagagatcacggagctcaggcgcacgctccaaggcctggagattgagctgcagtcccagctgagcatgaaagcggggctggagaacacggtggcagagacggagtgccgctatgccctgcagctgcagcagatccagggactcatcagcagcatcgaggcccagctgagcgagctccgcagtgagatggagtgccagaaccaagagtacaagatgctgctggacatcaagacacgtctggagcaggagatcgccacctaccgcagcctgctcgagggccaggacgccaagaagcgtcagcccccgtagcacctctgttaccacgacttctagtgcctctgttaccaccacctctaatgcctctggtcgccgcacttctgatgtccgtaggccttaaatctgcctggcgtcccctccctctgtcttcagcacccagaggaggagagagccggcagttccctgcaggagagaggaggggctgctggacccaaggctcagtccctctgctctcaggaccccctgtcctgactctctcctgatggtgggccctctgtgctcttctcttccggtcggatctctctcctctctgacctggatacgctttggtttctcaacttctctaccccaaagaaaagattattcaataaagtttcctgcctttctgcaaacataaaaa

SEQ ID NO: 13 - DNA, 1212 bp, Homo sapiens
  misc_feature (1)..(1212)
  misc_feature (1)..(1212): upregulated in mutant
atggcgtcccagccgccacctccccccaaaccctgggagacccgccgaattccgggagccggaccgggaccaggaccgggccccactttccaatctgctgatttgggtcctactttaatgacaagacctggacaaccagcacttaccagagtgcccccacctattcttccaaggccatcacagcagacaggaagtagcagtgtgaacacttttagacctgcttacagttcattttcttctggatatggtgcctatggaaattcattttatggaggctatagtccttatagttatggatataatgggctgggctacaaccgcctccgtgtagatgatcttccacccagtagatttgttcagcaagctgaagaaagcagcaggggtgcatttcagtccattgaaagtattgtgcatgcatttgcctctgtcagtatgatgatggatgctaccttttcagctgtctataacagtttcagggctgtattggatgtagcaaatcacttttcccgattgaaaatacactttacaaaagtgttttcagcttttgcattggttaggactatacggtatctttacagacggctacagcggatgttaggtttaagaagaggctctgagaatgaagacctctgggcagagagtgaaggaactgtggcatgccttggtgctgaggaccgagcagctacctcagcaaaatcttggccaatattcttgttctttgctgttatccttggtggtccttacctcatttggaaactattgtctactcacagtgatgaagtaacagacagcatcaactgggcaagtggtgaggatgaccatgtagttgccagagcagaatatgattttgctgccgtatctgaagaagaaatttctttccgggctggtgatatgctgaacttagctctcaaagaacaacaacccaaagtgcgtggttggcttctggctagccttgatggccaaacaacaggacttatacctgcgaattatgtcaaaattcttggcaaaagaaaaggtaggaaaacggtggaatcaagtaaagtttccaagcagcaacaatcttttaccaacccaacactaactaaaggagccacggttgctgattctttggatgaacaggaagctgcctttgaatctgtttttgttgaaactaataaggttccagttgcacctgattccattgggaaagatggagaaaagcaagatctttga

SEQ ID NO: 14 - DNA, 923 bp, Homo sapiens
  misc_feature (1)..(923): upregulated in mutant
aggcatgatggaggtcgagtcctcctactcggacttcatctcctgtgaccggacaggccgtcggaatgcggtccctgacatccagggagactcagaggctgtgagcgtgaggaagctggctggagacatgggcgagctggcactcgagggggcagaaggacaggtggagggaagcgccccagacaaggaagctggcaaccagccccagagcagcgatgggaccacctcgtcttgaatctgaccttgtccaagaaggctggacgagagaccttctgtcccctcccagaggggaaaccctggcactggcccagcagcctcttctctgagctccatgtcccagataaaccaggccagactgagaaggctccccagaggcctctgtggcctccactccgggaaagccctctgcccacacccacaggcttcacattcccaccaccttcgcaccgtgcccaggtacactttcaagacactgtaaccacaagatgttatttattgagctggcgccgggacttgggcggggcctgccctacagtgagcagcccacacaggaacgctcctctcgcgagcggcccgggcagggaccctgtcccaacaccaacacctcctctccagcccaatcttctgggtccagacctgcttgtcccttttttagaaaacacttttaaactttttaaaaattttaaaccttttttcagcagatatggagagagctgacaatcaattcacattttttaagccattttagctaaactgtcattgtgcatctctgaggttccctcatggagctccacagatccatttttagggaagggattttggctcaaaacgatctgaccacctctgccctgtccaccaggataagtgacacctaggacccaggaaataaatgccgatgatttgtgtgaaaaaaaaaaaaaaaa

SEQ ID NO: 15 - DNA, 3769 bp, Homo sapiens
  misc_feature (1)..(3769): upregulated in mutant
ggcggcggaggcagcagtcttagaatgagtagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactcccatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtccccccctttttttttacacatatttgcatatgtatgatagtgtgcatgtggttgtcatttttatttcaccaccataaaacccttgagcacaacagcaaataagcaggacgggcccaaagttatttatgattctggggggaaaataacccaaaggcatgctccagacataaatagctcactgc

SEQ ID NO: 16 - DNA, 2281 bp, Homo sapiens
  misc_feature (1)..(2281): upregulated in mutant
ggacagaaaactccctccttttccaagttagccttatagtctagggcttaaaatactggtttaatggtgaaggtaagtgcttttcttctttttgggtagaaggattattactaacttaccaaaggtccattaaggggagggaacagttttaggagaagtcagagaaaagacattaacagcaacataaggatctccatctggtaatattgcctaattccaaaatgaagagactctctgaaaaagataactgattcaatgaagaccctagggcaaggcttgagaagccactggtaccaatggacactgtggacaatggtcatttctccaaggacgctataaaagactgtcgtagtaaaagagattcagggcacagggaaactccaccacaaagcgtggtaccatttcccacagaagctaaatggacgggaagcctgccaccaggaaaggtccagatttctgttcattacgctatgggctggctcttatcatgcacttctcaaacttcaccatgataacgcagcgtgtgagtctgagcattgcgatcatcgccatggtgaacaccactcagcagcaaggtctatctaatgcctccactgaggggcctgttgcagatgccttcaataactccagcatatccatcaaggaatttgatacaaaggcctctgtgtatcaatggagcccagaaactcagggtatcatctttagctccatcaactatgggataatactgactctgatcccaagtggatatttagcagggatatttggagcaaaaaaaatgcttggtgctggtttgctgatctcttcccttctcaccctctttacaccactggctgctgacttcggagtgattttggtcatcatggttcggacagtccagggcatggcccagggaatggcatggacaggtcagtttactatttgggcaaagtgggctcctccacttgaacgaagcaagctcaccaccattgcaggatcagggtcagcatttggatccttcatcatcctctgtgtggggggactaatctcacaggccttgagctggccttttatcttctacatctttggtagcactggctgtgtctgctgtctcctatggttcacagtgatttatgatgaccccatgcatcacccgtgcataagtgttagggaaaaggagcacatcctgtcctcactggctcaacagcccagttctcctggacgagctgtccccataaaggcgatggtcacatgcctaccactttgggccattttcctgggttttttcagccatttctggttatgcaccatcatcctaacatacctaccaacgtatatcagtactctgctccatgttaacatcagagatagtggagttctgtcctccctgccttttattgctgctgcaagctgtacaattttaggaggtcagctggcagatttccttttgtccaggaatcttctcagattgatcactgtgcgaaagctcttttcatctcttgatatgcaagtttcctcatgggaatctcaaggggatttgggctcatcgcaggaatcatctcttccactgccactggattcctcatcagtcaggattttgagtctggttggaggaatgtctttttcctgtctgctgcagtcaacatgtttggcctggtcttttacctcacgtttggacaagcagaacttcaagactgggccaaagagaggacccttacccgcctctgaggacataaagttacaaacttaaatgtggtactgagcatgaactttttaaacattttttacttctctccatattcctgaccatagactcagcagttcttaactctggctgtgtgttagtcttccctggggagcctttataagacactgatacttgggacccactccagagattctgaatgaattggtctggggtggaacccagatactactaatttttagatactccttagaggtttctagcatgcgcccggggttgacaacagctggacaaacttgaaaagtcaattcatgtggcctttgaattttcctcattggaaagtactaaataaataaaaattcatgtgaaaatgatcactgataaatatcttcatggtggggcaggttattggatgcagagaagatctgctcggaattgtagccatatgttacagatctcagcaccgatcagaactgtaaagctataatccccagaattaaagtttttattattttttatacattgtaaaacatagacgtttatttatgtgattaaattctattaaaatttacatgctaaaataaaaaaaaaaaaaaa

SEQ ID NO: 17 - DNA, 4370 bp, Homo sapiens
  misc_feature (1)..(4370): upregulated in mutant
tggggccgtcgtgccccgccccagtgcggccggggcgcgggttcgagctgctgctcggcaagcctgggtgtctagggcatgagcggagtgtggggggccggcgggcctcggtgccaggaggcgctcgcggtcctcgcctcgctgtgccgggcccggccgccccctctcgggctggacgtggagacttgtcggagcttcgagctgcagcccccagagcggagtcccagcgcggcaggcgcagctgtgctggatgccctggaggaacaaggaggcctccagtccctcttctgctaatccccccttggaagccctccagagccccagcttcagaggcaacatggcggacaagctgaaggaccccagcaccctgggcttcctggaggcggccgtgaagatcagccacacgtccccagatatcccagctgaggtgcagatgtcggtcaaggagcacatcatgcgtcacacccggctgcagcggcaaactacagagccagcgtcatccaccaggcacacgtccttcaagcgccacctgccaaggcagatgcatgtctccagtgtagactatggcaatgagcttccaccagcagcagagcagcccaccagcattggccgcatcaagcctgagctctacaagcagaagtcggtggatggggaggatgccaagtctgaggccaccaagagctgcgggaagatcaacttcagcctacgctacgattacgagaccgagaccctgattgtgcgtatcctgaaggcttttgacctccctgccaaggacttttgtggaagctctgacccttatgtcaagatctacctcctgcctgaccgcaaatgcaagctgcagacccgggtgcaccgcaagaccctgaaccccacctttgatgagaacttccacttccctgtgccctatgaggagctggctgaccgcaagctgcatctcagtgtcttcgactttgaccgcttctcccgccatgacatgattggcgaggtcatcctggacaacctctttgaggcctctgacctgtctcgggaaacctccatctggaaggatatccaatatgccacaagtgaaagcgtggacttgggagagatcatgttctccctttgctacctgcccactgcaggcaggctcaccctcacagtgattaagtgtcggaacctcaaggcgatggacatcacaggctattcagatccctatgtgaaagtgtccttgctctgtgatgggcggaggctgaagaagaagaaaacaaccataaagaaaaacactctcaatcctgtctacaatgaggccatcatctttgacattcccccggaaaacatggatcaagtcagcctgctcatctcagtcatggactatgatcgagtgggccacaatgagatcataggagtctgtcgtgtggggatcactgctgaaggcctgggcagggaccactggaacgagatgctggcatacccccggaagcccatcgcacactggcactccttggtggaggtaaagaaatccttcaaagagggaaaccctcggttgtgatttcattcacgtggatgctgcaagcagagagactgccacctggagttaggatggcagggccgagctgctagcttcgacagtgagagctcgtgcccatctccgaaaccacctccaacaccatgagatgtgcagccaaataacacaaatgggactcagcaatgttctctttgcacttgttcaaccgtctaaacagtgttgtgcagtcgcagtggcggcagcagcggcagccgtccgtcactccagagtcttacctgctcctgtgtaggtcaaagctgagacacttgtcatgtggtcagatctgtcttagtcttttgtgtctcccaaggtgatgtttttgtggtttttcactttttatgggtctgctggtaagaaaagggaaaaaaaaaaagagtaggaaaagacaaagggtggaaagcccagaagttggttctagaaaatacaaggtacagttttttggaaagaggtggattgagtttctgaaatgctgcgtctccagcaagctccacaaccctggaaaaggaggaggcgtggtgggactgcggctgcattttcagtgccctgcaggaatggggtgaatccatggaagagcctttgtgaggtgaattactagtctttgaagctcttcgctggcaggacataaaccacgctgggaccctcagtcccgtgttgaaatcacaagacctctgaggcgcttgtgagccttcagctgatgtgcagggttcctcgctccccttcctcgctggctttccccatgaattgaaaacgtcttgattcctcatgggaacttagtcactttctttaggaatgattcagttcagccgaaactaaacacgccatatgttttctgatgcgccacagttaatgatcacatttgtttgaaaacaggatctttgtcctcagaaataatcttgattgtaaagtaagagagaaacatatgttctggaatgatttctgcattatacatagctttataattccatgaagtatcaagctgctgtttaaaatgcctaaacagattaaagaaaaacctagattaaatgttcttttccaaatggggagttttgaaatccaaatagcagtttgagtccatctttctatgaagagtatgtttttgattgaaaccgagtcagcgtaagcctatttcagcccctttggctctttgacaatattcatcttaaactttagttctataaattactaagtacttcagtgttgtttgtttcagtggttgtgagggtttcttgtttcctttagatctttaccttaggccaaactcagtttaggaaaaaaaaaattgtttgaactattctgtgttgtaatagcacatttctgttgagttaatagtggtcttcccaccaacccctaaaaccaggttgatttgctcaatctcagtttgtataaccccagtaagcgcatggaggtgagtttctacttcgacttgtggcgacacagtgccagtttcctgagctgtgtgtactacttgcaagaggcaagtctgctgaagaaccccttcttttaacagtggaaaatcaagaacagaaaatctgttcacacacctgtgtcttattctttatatatgctacatggtagaggagcagcgaccaataaagtatccctcattttaaaccaagttgtcagatttctctgctgatagaacgctgcatagtatcttctagccctaaaaatgaccaggttgggcatgagttaacaggatattgtggcaaaaggaattttctgcttagaaaagtaaatgtagataaacctaaactccaccctgtactgagccttaggctagggactcatgtgtaaagatgacttttctccccagatatcttgtcattgacaggccttcctagtctggtagtcctacatttgggtgttctcctgagagtgagaaaaaacccaaggtcatgacaagattcctggcaggtgacagacttcattatgattctgactctagactgaccccatcaagatagtcgtttccgtcagtgaacagatgctgaaggccttgccattacaaaggagacaactgagttccctgggtcataaggagtccagaaattagtgtgagaaggtccttctggaagtgaactcaaaattcatgtccagctgtcatcgttaaacccttaggcaggtgagtataacccattctaacctcttggagaagggagtgtgatcaatgtgaatcacaatagtattgctcatctgagacattcctcttatatccaagcagctatctcccatgagtgaacatcccagtggtttcagaaaatcatgtcctgaacagattctagccagccccacaccaccctggccaactgcaaccgctgctgctctagcagtctgggtcaattcctacaggcttgggagcccaccccagggaggcttttatctcctgcagcctcatccccagcaccctcaaggtagactactcctcagcctgaatcatgaatgaaacctgaacatctgccaacttcattatctagcccagtatctcttccctcacagagaagaagaaaatgaaatacaaagttgatccatttgttggctttctctggccggagaaacctctcgggttgttgctgtgagctttgatgaagttgtgataaccagggccatttgcacataggtttttttaaaaaacatgaaaagttcccttctcctttatttagcagtgtttccgggctgaaagacgccctcctccccctctgcgcttacccaggacgttaattaattgcactgtcatttaaaatattatgttgttaattactcgtttgtgtcttcccacccgtgtcacagcctggagtttttattcaattaaatctctgtctcttgccca

SEQ ID NO: 18 - DNA, 1693 bp, Homo sapiens
  misc_feature (1)..(1693): upregulated in mutant
gcccagcagcccagccacagaggagccagcgaacctctcccggcgcctgttctgggggctttctgttccagcgtcaaggatggaggagggggagaggagccccttactgtcccaggaaactgcaggccagaagcccctctctgtgcacaggccacccacctcaggctgcctaggtccagtgcccagggaggaccaggcggaggcctggggctgcagctgctgtcccccggagaccaagcaccaggccttgagtggcactcccaagaaaggaccagccccttccctctccccagggagcagctgcgtcaagtatctgatcttcctctccaacttccccttctccctgctggggctgctggccctggccatcgggctctggggcctggctgtcaaggggtctctgggaagtgatctgggggggcccctgcccacagaccccatgctggggctggcactgggagggctggtggtcagcgcagcgagcctggctggctgcctgggcgccctctgcgagaacacctgcctgttacgtggcttctccgggggcatccttgccttcctggtgcttgaggccgtggcgggggccctggtggtggccctctggggcccgctgcaagacagcctggagcacaccctgcgtgtggccatcgcccactaccaggacgacccagacctgcgcttcctcctcgaccaagtccagctcgggctgaggtgctgcggagctgcctcctaccaggactggcagcagaacctgtactttaactgcagctcccccggggtgcaggcctgcagccttcccgcctcctgctgcatcgacccccgcgaagatggagcctctgtcaacgaccagtgcggcttcggggtcctgcgcctggatgcggacgcagctcagagagtggtgtacctggagggctgcggcccgccgctccggcggtggctgcgcgcgaacctggctgcctcgggcggctacgcaatcgcggtggtgctgctgcagggcgcggagctcctgctggccgcccggctactcggggccctcgctgcccgcagtggggcggcgtacggccccggagcgcgcggggaggaccgcgctggcccccagagccccagccccggcgccccgcccgctgccaaacccgcccggggctgagcgcacgccccgaggtccgagaccgccacgcacagggatacagggggcgcctccgcccggctaaaaagcgctgcctgcgccgccgccgccgcctgatttcgctcgggcttcgggtgacttcgccgcaggacctacccagctcgctcacttcgctcgctccgcgtcccccatgccagcccccaacgcagggcgcccggcgaagccacgggactggcgggaggagcacgcggggccggaggaaatcctggagctgaccctcacctccgagcccccactcccaccccagccgcacagttcccacctcctggcacctccctcccctggggccgccaccccttctgggctcgtgatggtggagctaaggtccaggcctctccctcccgagtgcatttttggggagatagtaaatgttttattcgggtgtatcattcatacagtaaagacaccaatcttcacaggcaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 1261 bp, Homo sapiens
  misc_feature (1)..(1261): upregulated in mutant
ccctcccttccgagctgagcttaccctgggcgcaaacgagcgaggcaggggcgcgagtggaagctggagttccggggtgggcggggaggcgactgtccgtggtgctgagcgccggcgagagcgggcgcggagcggctgatcggctccctcgaactggggaggtccagtggggtcgcttagggcccaaagcccccacccggctccaaaagctcccagggcctccccaggcaccggtgctcggcccttccttcggtcagaaagtcgccccctgggggcagttcgtcccaaagggtttcctcgaaagaatctgagagggcgcagtccttgaccgagggaatctctctgtgtagccttggaagccgccagccccagaagatgcctgccttcaatagattgtttcccctggcttctctcgtgcttatctactgggtcagtgtctgcttccctgtgtgtgtggaagtgccctcggagacggaggccgtgcagggcaaccccatgaagctgcgctgcatctcctgcatgaagagagaggaggtggaggccaccacggtggtggaatggttctacaggcccgagggcggtaaagatttccttatttacgagtatcggaatggccaccaggaggtggagagcccctttcaggggcgcctgcagtggaatggcagcaaggacctgcaggacgtgtccatcactgtgctcaacgtcactctgaacgactctggcctctacacctgcaatgtgtcccgggagtttgagtttgaggcgcatcggccctttgtgaagacgacgcggctgatccccctaagagtcaccgaggaggctggagaggacttcacctctgtggtctcagaaatcatgatgtacatccttctggtcttcctcaccctgtggctgctcatcgagatgatatattgctacagaaaggtctcaaaagccgaagaggcagcccaagaaaacgcgtctgactaccttgccatcccatctgagaacaaggagaactctgcggtaccagtggaggaatagaacaggagcagtgtgacatgaggtggcctgaacacctgagggactggacatcccatgttcagcaatgtcaatggcatcaggagggcgccccaagggccccatcgcttcccttcatgcatccattgttctgttcattcattcatccatacatccacctgcctctgagctttcacctctgactccctaactccatcagacctctacgcaccataagactctgccagaactgagaagccgg

SEQ ID NO: 20 - DNA, 1506 bp, Homo sapiens
  misc_feature (1)..(1506): upregulated in mutant
aggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccatcaggcagtggctgttggaaagatttaagatttcacacctatgacatacatgggatagcacctgggccgccatgcactcaataaagaatgtatttt

SEQ ID NO: 21 - DNA, 2955 bp, Homo sapiens
  misc_feature (1)..(2955): upregulated in mutant
atgactgttttctgcctcctgggtctcaaaggcaggtgcaatgcccagaggctggtcctgcacagagggacccagggcgtgctggccttcccagtgtcccactggtggctgatctaagcagtgctttccctcgttttttttattttgagacagaatcttgctctgtcccccaggctggagagcaacggcacgatctcggctcactgcagcctcccgcttcggcctcccagatagctgggattacaggcatgtgccctcatgcctggctaatttttgtatttttagtagagacggggtttcaccttgttggtcaggctggtctccagttcctggcctcaagtgatccgcccgcctcggcctcccaaagtgctgggacgacaggcgtaagcctctgcgcccggcctcctcattgtttctaatggtccctcagtgcccaccgggtgatggtggcagattttccatccgcattgctcccggacatgcggacgtaggtttcagcagttcccatcattagacacagttgccgtaggctgggttcttagtagcaggctcagtggggcctttgagtctttccatggctggtgcccgccggactcactgagggaaactaatggggaagcactggtctccattccctgcacgcacagcccactgctgcgatggtgaagggataaggttgtgttcacgcgtcgaagtttgtgtccactgcacacgcggctcactgctgcgatggtggagggataaggtcgtattcacgcgttgaagttggtgcctctcagtatcgctttatgtttgttggccacttggtggtccttgtatgagctgtccactccttgttgattggccagagctctttacatggggaggaccatgggccggaccatgggccctcatccagcaggtgtgcaggcacccacgcactctcttgtcccttgcagcggctggctgtcggcaattggattcttccagtacagcccgggcgctgccgtggtcatgctgcttccagccatcatgttctccgtgtcggctgccatgatggccatcgcgatcatgaaggtgcacaggatctaccgaggggctggcggaagcttccagaaggcacagacggagtggaacacgggcacttggcggaacccaccgtcgagggaggcccagtacaacaacttctcaggcaacagcctgcccgagtaccccactgtgcccagctacccgggcagtggccagtggccttagagggagcctgccctgcccccaccgcccaccacctcctccccttcattcctgctgctacccctggtcccgagggctgggagtacctggggccccatccccccagctgggatggtggaagccggtggtggccacggaccgcccccctcctgccagggccacagaacccgtgttcatctcatccgagagcggagttcctcacaagcactccccagcagcccttggcctctgccgtccacaggacgccctcttgctcccggaaacgtgtggtcacccgccgtccactgcacggctggtacggccttgtcttcaggtctcgaggcctgactccgggggacaggtggcagcaggtcggccgccctcccgtcctcccagagctgctggcgctgaggtcagagcgggtctgatggggagctccgtctcaccggccacccgccgtcaccatggcagatgcccttggccggaactaataagaggcgtcggggccagcttccggtcccctgcagtgatagagggcttggtgcctagctgagtcctcgctgtccccgccatcccctgatctgtgcggctccagcctcgccccctccccacgtgcaccatacctggggagttcctggtccagggtatcctggggccaccctccctgcctccaaaacagggatccctggcaggctgtctttccacgcccctgagttcagagtcggggacccaggccaggtcgggagcacagccgctccccaaacccagcaaaccggcagagagccggtttcccagcagccggagccctgcaggagaggcctttgtgttttgttttgttttgttttttctcttttgagacagagtttcactctgtcgcccaggctggagtgcagtggtgtgatctcggctcactgcaacctctgcctcccgtgttcaagcagttctcctgcctcagcctcccaaatagctgggattacagttgcctgccaccacgcccagctaatttttatatttttagtacagatggggtttcaccatgttggccaggctggtctcgaactcctgacctcaagtgatccacccgccttggcctcccaaagtgctgggataataggtgtcagccaccgcacccagcctggagtggccttttatgagaggggacccgtcaaatctgtgccttatggaggggtccggcagcggccacaattgtcttgtcccctcaccccccaactccccctggaacacctctcccaggcaagacattttcacagcaccattcacaacggttgggccaaaaagaaacttttccttcatcatttcctgcactcgctgaccacaactttggacaccccaggctgccacccctccccacccgttcacccccaggatgctgttgctgtaggacgcctgctgccctggagccctccccaggatgtgagccagtcccctcgctggtacggaatgccgctgggtgcccggaggcggccatggtgtctcgatggacggcagccaggatggagcacccatgggtctcacggccatgcttcagggtcttcaggtcctgcccccggccagtctgccaagaggcacccccttccccagcctctcgcctgcactgatgcagacaaaatctcacctggcaggcccaacccccaccccacccctcccccgccgtgtgtggcccctcgccgcatcgttggggttttgttacgtgaaaatatcctggaaataaatacatgtttctgc

SEQ ID NO: 22 - DNA, 1177 bp, Homo sapiens
  misc_feature (1)..(1177): upregulated in mutant
ccgcgcggacggtgggcaggcgacggcggcgtgtggatggaggccttcgcccttggcccagcgcggcggggcaggcggcggacccgggccgccggctccctgctctctcgggccgccatcctcctctttatctccgccttcctggtgcgggtgccctcatcagttggacacttggttcgattaccaagagcttttcgcttgaccaaagattcagtgaaaatagtgggatcaacaagttttccagtgaaagcgtatgtcatgctccatcaaaagagtccacacgtgttatgtgtaacgcaacaactgcgaaatgctgaactgatagacccatcattccaatggtatgggcctaaaggaaaagttgtttcagtagaaaaccgcactgcacaaataacatccacaggaagccttgtattccaaaattttgaggagagtatgagtggaatttatacatgtttcctcgaatataaacctactgtggaagaaattgttaaacgtcttcaactaaaatatgctatatatgcttatcgtgagcctcattattattatcagttcacagctcgatatcatgcagtcccctgcaatagcatttataatatttcttttgagaagaaacttcttcagattttaagcaaactgcttcttgacctttcatgtgaaatttccttacttaagtctgaatgccatcgcgttaaaatgcaaagagctggtttgcaaaatgaattgttctttgcattttcagtttcatctctagacactgaaaaaggacccaagcgatgtacagaccataactgtgaaccttacaaaagactttttaaggctaaaaatctcatagagagattttttaatcaacaagtagaaattcttggcagacgtgcagaacaattacctcaaatatactatattgaaggtactctccaaatggtttggattaatcgctgctttccaggatatggaatgaatgtccagcaacatccaaaatgtcctgagtgctgtgtgatctgcagccctggatcatataacccccgtgatggaattcattgccttcaatgcaatagcagcctggtgtatggagcaaaaacgtgcttataagccattatcttcagttattcagtggtttattaaatgcaaagtatatatttgaaatattaacataataaattattatgccaaaatt

SEQ ID NO: 23 - DNA, 1195 bp, Homo sapiens
  misc_feature (1)..(1195): upregulated in mutant
cgcgccggagcgggaccgacgggaccgagcgagcgaccgacgcgccacccgccgacgcctcagccgcttggggcccgcacggaccctctacttcagtgtagaatgagccaaggagactcaaacccagcagctattccgcatgcagcagaagatattcaaggagatgaccgatggatgtctcagcacaacagatttgttttggactgtaaagacaaagagcctgatgtactgttcgtgggagactccatggtgcagttaatgcagcaatatgagatatggcgagagcttttttccccacttcatgcactgaattttggaattgggggagatacaacaagacatgttttgtggagactaaagaatggagaactggagaatattaagcctaaggtcattgttgtctgggtaggaacaaataaccacgaaaatacagcagaagaagtagcaggtgggatcgaggccattgtacaacttatcaacacaaggcagccacaggccaaaatcattgtattgggtttgttacctcgaggtgagaaacccaatcctttgaggcaaaagaacgccaaggtgaaccaactcctcaaggtttcgctgccgaagcttgccaacgtgcagctcctggataccgacgggggttttgtgcactcggacggtgccatctcctgccacgacatgtttgattttctgcatctgacaggagggggctatgcaaagatctgcaaacccctgcatgaactgatcatgcagttgttggaggaaacacctgaggagaaacaaaccaccattgcctgactggctcttatcagtgttaatagcatctcagcttcctcagatcagttctatcactggcactacagaatccttctctttcttaaggcactttgcattgtagaatgttcctggatgttcatatctagtgtttgaaggggaggagggatttaaactggtcctgtacatagaaggtttgtttgacagaggagaaaaattagccaaggaagattgttgtttaaattcatttgaaaccagaaggggactttttagttgtatgtgtaacacattcattgaattattatcactgttttcttgggacaacatcaagcctaaatactgaacaatatgaagattcttttcttggcctttctgtggattatgtcatatataataattatcagaatcattctacttggctttttcc

SEQ ID NO: 24 - DNA, 1853 bp, Homo sapiens
  misc_feature (1)..(1853): upregulated in mutant
ctgagcctctccgtgcaatgattaacccggcggggcggccggcgcgggacccgagacggaggcgcggggccggggcgggaccccgcaggaccgctcggcttcctgctctcgccggagtttccgcgtagagggcgcatcgccggcccggggcccttggtgcggcgtggcgcagggcgcggcgtggggcgcgcgtgggcgcggcgcaggcggcccgggtcaccatgaggactctccgcaggttgaagttcatgagttcgcccagcctcagtgacctgggcaagagagagccggccgccgccgcggacgagcggggcacgcagcagcgccgggcctgcgccaacgccacctggaacagcatccacaacggggtgatcgccgtcttccagcgcaaggggctgcccgaccaggagctcttcagcctcaacgagggcgtccggcagctgttgaagacagagctggggtccttcttcacggagtacctgcagaaccagctgctgacaaaaggcatggtgatccttcgggacaagattcgcttctatgagggacagaagctgctggactcactggcagagacctgggacttcttcttcagtgacgtgctgcccatgctgcaggccatcttctacccggtgcagggcaaggagccatcggtgcgccagctggccctgctgcacttccggaatgccatcaccctcagtgtgaagctagaggatgcgctggcccgggcccatgcccgtgtgccccctgccatcgtgcagatgctgctggtgctgcagggggtacatgagtccaggggcgtgactgaggactacctgcgcctggagacgctggtccagaaggtggtgtcgccatacctgggcacctacggcctccactccagcgaggggcccttcacccattcctgcatcctggaaaagcgcctcctccgccgctcccgctcgggggacgtgctggccaagaaccctgtggtgcgctccaagagctacaacacgcctctgctgaaccccgtgcaggagcacgaggcggagggcgcggcggccggcggtaccagcatccgcaggcactctgtgtcggagatgacgtcctgccccgagcctcagggcttctccgacccgcccggccagggccccaccgggaccttcaggtcctccccggcgccccactcagggccctgccccagcagactgtaccccacgacccagccccctgagcagggcttggatcccacccgcagctccctgccccgctccagcccggagaacctggtggaccagatcctggagtccgtggactcggattctgaagggattttcattgactttggccggggccggggctctggcatgtccgacttggagggctctgggggccggcagagtgtcgtgtgaggcctcacagctggccttgagtttttactgacacgtccctgtgtgcgggggtgtccatgtggcgtgtgtgtgagtgagacttttttactgcgtcccgtcccgccagccctatcggcctcgtcactggccttggtcactttgtatttctgtcttggttggaaataccatcagccttccttgctcggcccaggtctgtttcaggcatctgagtcggcgtttacccaggggccgggccagagacgggggtcggccgctcgctcccacgctcctcctgccccagccctctggtgtccacacctgcccacagagaatgtaaacccagtgggctctgcccacgccgggccccaaagtgaccagactccagcacacctgtctcctcctgcctggggtggccatggggatggaagggggtggaataaaacctgtcaccctgggaaaaaaaaaaaaaaaaaaaaaaaaaaaaaa

SEQ ID NO: 25 - DNA, 2661 bp, Homo sapiens
  misc_feature (1)..(2661): downregulated in mutant
gctcccggggccacgggatgacgcctcctccgcccggacgtgccgcccccagcgcaccgcgcgcccgcgtccctggcccgccggctcggttggggcttccgctgcggctgcggctgctgctgctgctctgggcggccgccgcctccgcccagggccacctaaggagcggaccccgcatcttcgccgtctggaaaggccatgtagggcaggaccgggtggactttggccagactgagccgcacacggtgcttttccacgagccaggcagctcctctgtgtgggtgggaggacgtggcaaggtctacctctttgacttccccgagggcaagaacgcatctgtgcgcacggtgaatatcggctccacaaaggggtcctgtctggataagcgggactgcgagaactacatcactctcctggagaggcggagtgaggggctgctggcctgtggcaccaacgcccggcaccccagctgctggaacctggtgaatggcactgtggtgccacttggcgagatgagaggctacgcccccttcagcccggacgagaactccctggttctgtttgaaggggacgaggtgtattccaccatccggaagcaggaatacaatgggaagatccctcggttccgccgcatccggggcgagagtgagctgtacaccagtgatactgtcatgcagaacccacagttcatcaaagccaccatcgtgcaccaagaccaggcttacgatgacaagatctactacttcttccgagaggacaatcctgacaagaatcctgaggctcctctcaatgtgtcccgtgtggcccagttgtgcaggggggaccagggtggggaaagttcactgtcagtctccaagtggaacacttttctgaaagccatgctggtatgcagtgatgctgccaccaacaagaacttcaacaggctgcaagacgtcttcctgctccctgaccccagcggccagtggagggacaccagggtctatggtgttttctccaacccctggaactactcagccgtctgtgtgtattccctcggtgacattgacaaggtcttccgtacctcctcactcaagggctaccactcaagccttcccaacccgcggcctggcaagtgcctcccagaccagcagccgatacccacagagaccttccaggtggctgaccgtcacccagaggtggcgcagagggtggagcccatggggcctctgaagacgccattgttccactctaaataccactaccagaaagtggccgtccaccgcatgcaagccagccacggggagacctttcatgtgctttacctaactacagacaggggcactatccacaaggtggtggaaccgggggagcaggagcacagcttcgccttcaacatcatggagatccagcccttccgccgcgcggctgccatccagaccatgtcgctggatgctgagcggaggaagctgtatgtgagctcccagtgggaggtgagccaggtgcccctggacctgtgtgaggtctatggcgggggctgccacggttgcctcatgtcccgagacccctactgcggctgggaccaaggccgctgcatctccatctacagctccgaacggtcagtgctgcaatccattaatccagccgagccacacaaggagtgtcccaaccccaaaccagacaaggccccactgcagaaggtttccctggccccaaactctcgctactacctgagctgccccatggaatcccgccacgccacctactcatggcgccacaaggagaacgtggagcagagctgcgaacctggtcaccagagccccaactgcatcctgttcatcgagaacctcacggcgcagcagtacggccactacttctgcgaggcccaggagggctcctacttccgcgaggctcagcactggcagctgctgcccgaggacggcatcatggccgagcacctgctgggtcatgcctgtgccctggccgcctccctctggctgggggtgctgcccacactcactcttggcttgctggtccactagggcctcccgaggctgggcatgcctcaggcttctgcagcccagggcactagaacgtctcacactcagagccggctggcccgggagctccttgcctgccacttcttccaggggacagaataacccagtggaggatgccaggcctggagacgtccagccgcaggcggctgctgggccccaggtgcgcacggatggtgaggggctgagaatgagggcaccgactgtgaagctggggcatcgatgacccaagactttatcttctggaaaatatttttcagactccctcaaacttgactaaatgcagcgatgctcccagcccaagagcccatgggtcggggagtgggtttggataggagagctgggactccatctcgaccctggggctgaggcctgagtccttctggactcttggtacccacattgcctccttcccctccctctctcatggctgggtggctggtgttcctgaagacccagggctaccctctgtccagccctgtcctctgcagctccctctctggtcctgggtcccacaggacagccgccttgcatgtttattgaaggatgtttgctttccggacggaaggacggaaaaagctctatttttatgttaggcttatttcatgtatagctacttccgactgcc

SEQ ID NO: 26 - DNA, 4000 bp, Homo sapiens
  misc_feature (1)..(4000): downregulated in mutant
gcacgatctgttcctcctgggaagatgcagaggctcatgatgctcctcgccacatcgggcgcctgcctgggcctgctggcagtggcagcagtggcagcagcaggtgctaaccctgcccaacgggacacccacagcctgctgcccacccaccggcgccaaaagagagattggatttggaaccagatgcacattgatgaagagaaaaacacctcacttccccatcatgtaggcaagatcaagtcaagcgtgagtcgcaagaatgccaagtacctgctcaaaggagaatatgtgggcaaggtcttccgggtcgatgcagagacaggagacgtgttcgccattgagaggctggaccgggagaatatctcagagtaccacctcactgctgtcattgtggacaaggacactggtgaaaacctggagactccttccagcttcaccatcaaagttcatgacgtgaacgacaactggcctgtgttcacgcatcggttgttcaatgcgtccgtgcctgagtcgtcggctgtggggacctcagtcatctctgtgacagcagtggatgcagacgaccccactgtgggagaccacgcctctgtcatgtaccaaatcctgaaggggaaagagtattttgccatcgataattctggacgtattatcacaataacgaaaagcttggaccgagagaagcaggccaggtatgagatcgtggtggaagcgcgagatgcccagggcctccggggggactcgggcacggccaccgtgctggtcactctgcaagacatcaatgacaacttccccttcttcacccagaccaagtacacatttgtcgtgcctgaagacacccgtgtgggcacctctgtgggctctctgtttgttgaggacccagatgagccccagaaccggatgaccaagtacagcatcttgcggggcgactaccaggacgctttcaccattgagacaaaccccgcccacaacgagggcatcatcaagcccatgaagcctctggattatgaatacatccagcaatacagcttcatcgtcgaggccacagaccccaccatcgacctccgatacatgagccctcccgcgggaaacagagcccaggtcattatcaacatcacagatgtggacgagccccccattttccagcagcctttctaccacttccagctgaaggaaaaccagaagaagcctctgattggcacagtgctggccatggaccctgatgcggctaggcatagcattggatactccatccgcaggaccagtgacaagggccagttcttccgagtcacaaaaaagggggacatttacaatgagaaagaactggacagagaagtctacccctggtataacctgactgtggaggccaaagaactggattccactggaacccccacaggaaaagaatccattgtgcaagtccacattgaagttttggatgagaatgacaatgccccggagtttgccaagccctaccagcccaaagtgtgtgagaacgctgtccatggccagctggtcctgcagatctccgcaatagacaaggacataacaccacgaaacgtgaagttcaaattcaccttgaatactgagaacaactttaccctcacggataatcacgataacacggccaacatcacagtcaagtatgggcagtttgaccgggagcataccaaggtccacttcctacccgtggtcatctcagacaatgggatgccaagtcgcacgggcaccagcacgctgaccgtggccgtgtgcaagtgcaacgagcagggcgagttcaccttctgcgaggatatggccgcccaggtgggcgtgagcatccaggcagtggtagccatcttactctgcatcctcaccatcacagtgatcaccctgctcatcttcctgcggcggcggctccggaagcaggcccgcgcgcacggcaagagcgtgccggagatccacgagcagctggtcacctacgacgaggagggcggcggcgagatggacaccaccagctacgatgtgtcggtgctcaactcggtgcgccgcggcggggccaagcccccgcggcccgcgctggacgcccggccttccctctatgcgcaggtgcagaagccaccgaggcacgcgcctggggcacacggagggcccggggagatggcagccatgatcgaggtgaagaaggacgaggcggaccacgacggcgacggccccccctacgacacgctgcacatctacggctacgagggctccgagtccatagccgagtccctcagctccctgggcaccgactcatccgactctgacgtggattacgacttccttaacgactggggacccaggtttaagatgctggctgagctgtacggctcggacccccgggaggagctgctgtattaggcggccgaggtcactctgggcctggggacccaaaccccctgcagcccaggccagtcagactccaggcaccacagcctccaaaaatggcagtgactccccagcccagcaccccttcctcgtgggtcccagagacctcatcagccttgggatagcaaactccaggttcctgaaatatccaggaatatatgtcagtgatgactattctcaaatgctggcaaatccaggctggtgttctgtctgggctcagacatccacataaccctgtcacccacagaccgccgtctaactcaaagacttcctctggctccccaaggctgcaaagcaaaacagactgtgtttaactgctgcagggtctttttctagggtccctgaacgccctggtaaggctggtgaggtcctggtgcctatctgcctggaggcaaaggcctggacagcttgacttgtggggcaggattctctgcagcccattcccaagggagactgaccatcatgccctctctcgggagccctagccctgctccaactccatactccactccaagtgccccaccactccccaacccctctccaggcctgtcaagagggaggaaggggccccatggcagctcctgaccttgggtcctgaagtgacctcactggcctgccatgccagtaactgtgctgtactgagcactgaaccacattcagggaaatgcttattaaaccttgaagcaactgtgaattcattctggaggggcagtggagatcaggagtgacagatcacagggtgagggccacctccacacccaccccctctggagaaggcctggaagagctgagaccttgctttgagactcctcagcacccctccagttttgcctgagaaggggcagatgttcccggagatcagaagacgtctccccttctctgcctcacctggtcgccaatccatgctctctttcttttctctgtctactccttatcccttggtttagaggaacccaagatgtggcctttagcaaaactgacaatgtccaaacccactcatgactgcatgacggagccgagcatgtgtctttacacctcgctgttgtcacatctcagggaactgaccctcaggcacaccttgcagaaggaaggccctgccctgcccaacctctgtggtcacccatgcatcattccactggaacgtttcactgcaaacacaccttggagaagtggcatcagtcaacagagaggggcagggaaggagacaccaagctcacccttcgtcatggaccgaggttcccactctggcaaagcccctcacactgcaagggattgtagataacactgacttgtttgttttaaccaataactagcttcttataatgatttttttactaatgatacttacaagtttctagctctcacagacatatagaataagggtttttgcataataagcaggttgttatttaggttaacaatattaattcaggttttttagttggaaaaacaattcctgtaaccttctattttctataattgtagtaattgctctacagataatgtctatatattggccaaactggtgcatgacaagtactgtatttttttatacctaaataaagaaaaatctttagcctgggcaacaaaaaaa

SEQ ID NO: 27 - DNA, 1583 bp, Homo sapiens
  misc_feature (1)..(1583): downregulated in mutant
attccaggcttttatcagaagcaaaagtttatccctatccaaaaatgaaattatacaaattcatacatatggtaaagattctagctgcctcttaaacgagacaaattttcagcagaagggcccaaaacaaaatggtcctagtcctctgaagtaaaaatatttatgggaaaaatcagtttttgtaagaaagaaaaattaagaatagtctctctcccaggccatagagcttcctttgtccaatcaggttggagcatgccaggggagatcagcttgctcagaagtcactatctaacgaagaaaaacataaatcctttcagagaaaggacactcagaaagaacaggtgaggagtgtgtaacatcaaaccaaaccaacctcatctcaaccccactcagggcctctctgatgcaatcccaaagttagcagtggcaatgcaactttcagaccgggagagctctgactgcctgttggatactcgagtcacagtccatggggtgttttgttattggccaatgtctgttaaccattaactctcttcatgattgcaccagttagaccatttgccctgatggggggaaaaagaagaagaaaaaacaaaatagaagaaaaaaagtgacaagagaaagcaagctgctaatggccgatcccaacacactttctgggagacagtagatgaaatcacagcacagtgaacacatgctgcctaaaatcagccttgttggggagtaatcccaactgccacccccaagacacagggtccagtccacttatctatagtctgagatgttcaccaacaaaaatggattaaagaatcttggacacaaagggccagaagaaaaagcctgagatgatcggttctgtgatttgttgcagagtgataaagagtaaatggtgagtaagtgttgtgtccagtccacttatctatagtctgagatgttcaccaacaaaaatggattaaagaatcttggacacaaagggccagaagaaaaagcctgagatgatcggttctgtgatttgttgcagagtgataaagagtaaatggtgagtaagtgttgtgtccaaaggtggagactccatagtcggcagacaggagagggatggccagtccactggcaggtgctcagtagtcatccaacgtctcgatttcacccatgttgagtcgctccgatgatgcattgactgaaaatcctaataaactgggatctgctcggaccatcttctgctttttcttcttcttgccactctgcacagcctcaaaattggattgttggttgttgctttgattggtctgaaatactgaatggagtgtactgtggttcatcccccacacagagtcctgctgttgtggctgctgtggcggctgctgtggcggctgctgctgctgctgctgtggcagatgctgctgctgacgctgctggttggctttctgtttggcacggngctcaaggaactgcttgggaaactccttggcctcagaagtatctcctaaataggccctgatataatcatggacctcaataaggagattctacttcttttcaaggaaaggaaacaaattgtggggaacatccaaggttatttgccg

SEQ ID NO: 28 - DNA, 1887 bp, Homo sapiens
  misc_feature (1)..(1887): downregulated in mutant
gcttgaaatcgaattcgggattcgggggggacgcaccagggagggaggggtccaggcagctgggccgccgcggacacctagcggcttcagggtgaaccccgaccgcagccgtcgccgcctcgggcagagtttgcgcccttgctttgcgccccgctgcgaagccgggcgggcgatcggcgcgtgaaagcgccgcgcgggcgacctctgtcctagtctcctgctccccccgccccgcttgtcccgtgcccttgtgacccaggctttggcgccgtcgccaggccccgcaatgtagctgcccctgcgcctcggcggaggctcctgccccgcgagcgcccggggcccggagccggcctgggggctcagccgagctcgggcggggccggggcgcggtggcgatgcaccgggccgttagcgccaggagccaggcagctgaggcggggggcaagcctccctcggagagccgcgcccccggcccgcgtcccgccgcgatgctgttccacagtctgtcgggccccgaggtgcacggggtcatcgacgagatggaccgcaggcaagagcgaggctcccgcatcagctccgccatcgaccgcggcgacaccgagacgaccatgccgtccatcagcagtgaccgcgccgccctttgtggcggctgtggcggcaagatctcggaccgctactacctgctggcggtggacaagcagtggcacatgcgctgcctcaagtgctgcgagtgcaagctcaacctggagtcggagctcacctgtttcagcaaggacggtagcatctactgcaaggaagactactaccggcgcttctctgtgcagcgctgcgcccgctgccacctgggcatctcggcctcggagatggtgatgcgcgctcgggacttggtttatcacctcaactgcttcacgtgcaccacgtgtaacaagatgctgaccacgggcgaccacttcggcatgaaggacagcctggtctactgccgcttgcacttcgaggcgctgctgcagggcgagtaccccgcacacttcaaccatgccgacgtgcaggcggcgcgtgcacgcgcggcggccaagagcgcggggctgggcgcagcaggggccaaccctctgggtcttccctactacaatggcgtgggcactgtgcagaaggggcggccgaggaaacgtaagagtccgggccccggtgcggatctggcggcctacacacgtgcgctaagctgcaacgaaaacgacgcagagcacctggaccgtgaccagccataccccagcagccagaagaccaagcgcatgcgcacgtccttcaagcaccaccagcttcggaccatgaagtcttactttgccattaaccacaatcccgatgccaaggacttgaagcagctcgcgcaaaagacgggcctcaccaagcgggtcctccaggtctggttccagaacgcccgagccaagttcaggcgcaacctcttacggcaggaaaacacgggcgtggacaagtcgacagatgcggcgctgcagacagggacgccatcgggcccggcctcggagctctccaacgcctcgctcagcccctccagcacgcccaccaccctgacagacttgactagccccaccctgccaactgtgacgtccgtcttaacttctgtgcctggcaacctggaggccatgagcctcacagcccctcacaaacgactcttaccaaccttttctaatgactcgcaaccccctcaccccacaatttctttaaaaaagaaattatctttagtttgaattccaagtgtattttaaaatagaggctttgagcaactaactaaccacattttaggatctcgcctggaaacagaggtaaaaaaaagaagtgtgcgcccggctaatgcagcggtgtggaccggaattc

SEQ ID NO: 29 - DNA, 3015 bp, Homo sapiens
  misc_feature (1)..(3015): downregulated in mutant
atgagttctgaatgtgatggtggttccaaagctgttatgaatggcttggcatctggcagcaatgggcaagacaaagcaactgccgaccctttacgcgcacgctctatttctgctgttaaaatcattcctgtgaagacagtgaaaaacgcctcaggcctagttctccctacagacatggatcctacaaaaatctgcactgggaagggagcggtgactctccgggcctcgtcttcctacagggaaaccccaagcagtagccctgcgagccctcaggaaacccggcaacacgaaagcaaaccaggtgaatggaaactttcttccagtgctgatgccaatggaaatgcccagccctcttcactcgctgtcaagggctctagaagtgtgcatcccaaccttccttctgacaagtcccaggatgccacttcctccagtgcagcccagccggaggtaatagttgtccctctctacctggttaatactgacagagggcaagaaggcactgccagacctccaacacctctggggcctcttggctgcgtccccacaatcccagcgactgcctctgccgcctcacctctgaccttcccgactctagatgatttcattccccctcatctgcagaggtggccccaccacagccagcccgccagtgcttgtggctcctttgcccccattagccagacgccaccatccttctcaccaccacctccgctggtccctcctgccccggaggacctccgcagagtctcggagcctgacctcccgggagctgtctcaagtaccgattccagtcctctactaaatgaagtttcttcttcccttattggaactgattcccaagccttttcatcagaatgcaagccttcatccgcctatccctccacaacgattgtcaatcctacaattgtgctcttgcaacacaatcgagaacagcaaaaacgactcagtagcctttcagatcctgtctcagaaagaagagtgggagagcaggactcagcaccaacccaggaaaaacccacctcacctggcaaggctattgaaaaaagagcaaaggatgacagtaggcgggtggtgaagagcactcaggacttaagcgatgtttccatggatgaagtgggcatcccactccggaacactgagagatcaaaagactggtacaagactatgtttaaacagatccacaaactgaacagagacactcctgaagaaaacccttatttccctacgtacaaattccctgaacttcctgaaatccagcaaacttccgaagatgatgattcagatctgtactctcccagatactcattttccgaagacacaaaatctcccctttctgtgcctcgctcaaaaagagagatgagctacattgacggtgtgaaggtagtcaagaggtcggccacactacccctcccagcccgctcttcctcactgaagtcaagctcagaaagaaatgactgggaacccccagataagaaagtagatacaagaaaatatcgtgcagagcccaagagcatttacgaatatcagcctggcaagtcttccgttctgaccaacgaaaagatgagtcgggatataagcccagaagagatagatttaaagaatgaaccttggtataaattcttttcggaattggagtttgggaaaccgcctcccaaaaagatatgggattatactcctggagactgctctatccttcctagagaggatagaaagactaatctagacaaagaactcagcttctgccagacagagttagaggcagatttagaaaaaatggagacgcttaataaagcacccagtgcaaacgtgccacagagctcagccatcagccctactccgggaatttcttcagagactcctggatatatatattcttccaacttccacgcagtgaagagggaatcagacggggctcctggggatctcactagcttggagaatgagagccaaatttataaaagtgtcttggaaggtggtgtcatccctcttcaggacctgagtgggctcaagcgaccatccagctctgcttccactaaaaattcagaatcgccaagacattttataccagctgattacttggaatccacggaagaatttattcgaagacgtcatgatgataaagagaaacttttagcggaccagagacgacttaaacgcgagcaagaagaggctgatattgcagctcgacgccacacaggcgtcattccgacgcaccatcagtttatcactaatgagcgctttggggacctcctcaatatagacgatactgcaaaaaggaaatctgggtcagagatgagacctgccagagccaaatttgactttaaagctcagacactaaaggagcttcctctgcagaagggagatattgtttacatttataagcaaattgatcagaactggtatgaaggagaacaccacggccgggtgggaatcttcccacgcacctacatcgagcttcttcctcctgctgagaaggcacagcccaaaaagttgacaccagtgcaggttttggaatatggagaagctattgctaagtttaactttaatggtgatacacaagtagaaatgtccttcagaaagggtgagaggatcacactgctccggcaggtagatgagaactggtacgaagggaggatcccggggacatcccgacaaggcatcttccccatcacctacgtggatgtgatcaagcgaccactggtgaaaaaccctgtggattacatggacctgcctttctcctcctccccaagtcgcagtgccactgcaagcccacagcaacctcaagcccagcagcgaagagtcacccccgacaggagtcaaacctcacaagatttatttagctatcaagcattatatagctatataccacagaatgatgatgagttggaactccgcgatggagatatcgttgatgtcatggaaaaatgtgacgatggatggtttgttggtacttcaagaaggacaaagcagtttggtacttttccaggcaactatgtaaaacctttgtatctataa

SEQ ID NO: 30 - DNA, 1378 bp, Homo sapiens
  misc_feature (1)..(1378): downregulated in mutant
ggtagcagcatccaccgggcgggaggtcggaggcagcaaggccttaaaggctactgagtgcgccggccgttccgtgtccagaacctcccctactcctccgccttctcttccttggccgcccaccgccaagttccgactccggttttcgcctttgcaaagcctaaggaggaggttaggaacagccgcgcccccctccctgcggccgccgccccctgcctctcggctctgctccctgccgcgtgcgcctgggccgtgcgccccggcaggcgccagccatgtcgatgctgccgtcgtttggctttacgcaggagcaagtggcgtgcgtgtgcgaggttctgcagcaaggcggaaacctggagcgcctgggcaggttcctgtggtcactgcccgcctgcgaccacctgcacaagaacgagagcgtactcaaggccaaggcggtggtcgccttccaccgcggcaacttccgtgagctctacaagatcctggagagccaccagttctcgcctcacaaccaccccaaactgcagcaactgtggctgaaggcgcattacgtggaggccgagaagctgcgcggccgacccctgggcgccgtgggcaaatatcgggtgcgccgaaaatttccactgccgcgcaccatctgggacggcgaggagaccagctactgcttcaaggagaagtcgaggggtgtcctgcgggagtggtacgcgcacaatccctacccatcgccgcgtgagaagcgggagctggccgaggccaccggcctcaccaccacccaggtcagcaactggtttaagaaccggaggcaaagagaccgggccgcggaggccaaggaaagggagaacaccgaaaacaataactcctcctccaacaagcagaaccaactctctcctctggaagggggcaagccgctcatgtccagctcagaagaggaattctcacctccccaaagtccagaccagaactcggtccttctgctgcagggcaatatgggccacgccaggagctcaaactattctctcccgggcttaacagcctcgcagcccagtcacggcctgcagacccaccagcatcagctccaagactctctgctcggccccctcacctccagtctggtggacttggggtcctaagtggggagggactggggcctcgaagggattcctggagcagcaaccactgcagcgactagggacacttgtaaatagaaatcaggaacatttttgcagcttgtttctggagttgtttgcgcataaaggaatggtggactttcacaaatatctttttaaaaatcaaaaccaacagcgatctcaagcttaatctcctcttctctccaactctttccacttttgcattttccttcccaatgcagagatcaggg

SEQ ID NO: 31 - DNA, 1576 bp, Homo sapiens
  misc_feature (1)..(1576): downregulated in mutant
tcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctcctgcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttggggcctttatcatctgctggactcctggattggttttgttacttctagacgtgtgctgtccacagtgcgacgtgctggcctatgagaaattcttccttctccttgctgaattcaactctgccatgaaccccatcatttactcctaccgcgacaaagaaatgagcgccacctttaggcagatcctctgctgccagcgcagtgagaaccccaccggccccacagaaagctcagaccgctcggcttcctccctcaaccacaccatcttggctggagttcacagcaatgaccactctgtggtttagaacggaaactgagatgaggaaccagccgtcctctcttggaggataaacagcctccccctacccaattgccagggcaaggtggggtgtgagagaggagaaaagtcaactcatgtacttaaacactaaccaatgacagtatttgttcctggaccccacaagacttgatatatattgaaaattagcttatgtgacaaccctcatcttgatccccatcccttctgaaagtaggaagttggagctcttgcaatggaattcaagaacagactctggagtgtccatttagactacactaactagacttttaaaagattttgtgtggtttggtgcaagtcagaataaattctggctagttgaatccacaacttcatttatatacaggcttcccttttttatttttaaaggatacgtttcacttaataaacacgtttatgcctatcagcaaaaaaaaaaaaaaa

SEQ ID NO: 32 - DNA, 2197 bp, Homo sapiens
  misc_feature (1)..(2197): downregulated in mutant
caggaagcccacccagccccgccacgcagagcccagaaggaaagaaagcctcatgcctgagccgaggggagcaccatggatctgacaaaaatgggcatgatccagctgcagaaccctagccaccccacggggctactgtgcaaggccaaccagatgcggctggccgggactttgtgcgatgtggtcatcatggtggacagccaggagttccacgcccaccggacggtgctggcctgcaccagcaagatgtttgagatcctcttccaccgcaatagtcaacactatactttggacttcctctcgccaaagaccttccagcagattctggagtatgcatatacagccacgctgcaagccaaggcggaggacctggatgacctgctgtatgcggccgagatcctggagatcgagtacctggaggaacagtgcctgaagatgctggagaccatccaggcctcagacgacaatgacacggaggccaccatggccgatggcggggccgaggaagaagaggaccgcaaggctcggtacctcaagaacatcttcatctcgaagcattccagcgaggagagtgggtatgccagtgtggctggacagagcctccctgggcccatggtggaccagagcccttcagtctccacttcatttggtctttcagccatgagtcccaccaaggctgcagtggacagtttgatgaccataggacagtctctcctgcagggaactcttcagccacctgcagggcccgaggagccaactctggctgggggtgggcggcaccctggggtggctgaggtgaagacggagatgatgcaggtggatgaggtgcccagccaggacagccctggggcagccgagtccagcatctcaggagggatgggggacaaggttgaggaaagaggcaaagaggggcctgggaccccgactcgaagcagcgtcatcaccagtgctagggagctacactatgggcgagaggagagtgccgagcaggtgccacccccagctgaggctggccaggcccccactggccgacctgagcacccagcacccccgcctgagaagcatctgggcatctactccgtgttgcccaaccacaaggctgacgctgtattgagcatgccgtcttccgtgacctctggcctccacgtgcagcctgccctggctgtctccatggacttcagcacctatggggggctgctgccccagggcttcatccagagggagctgttcagcaagctgggggagctggctgtgggcatgaagtcagagagccggaccatcggagagcagtgcagcgtgtgtggggtcgagcttcctgataacgaggctgtggagcagcacaggaagctgcacagtgggatgaagacgtacgggtgcgagctctgcgggaagcggttcctggatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcttccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtgacaagaagttcagcctcaagcatcagctggagacgcactatagggtgcacacaggtgagaagccctttgagtgtaagctctgccaccagcgctcccgggactactcggccatgatcaagcacctgagaacgcacaacggcgcctcgccctaccagtgcaccatctgcacagagtactgccccagcctctcctccatgcagaagcacatgaagggccacaagcccgaggagatcccgcccgactggaggatagagaagacgtacctctacctgtgctatgtgtgaagggaggcccgcggcggtggagccgagcggggagccaggaaagaagagttggagtgagatgaaggaaggactatgacaaataaaaaaaaaaaaggaattc

SEQ ID NO: 33 - DNA, 2907 bp, Homo sapiens
  misc_feature (1)..(2907): downregulated in mutant
gcgggacgtggacggaagaaaaaagagagtgagcgagcgccggaatcagcccggcgtggagtgcggacccgcggtagtgccagggggcgaaggcggcggtggtgaggaagatactttggttagtgaccacatcgcagcatgttgagtcaagtttaccgctgtgggttccagcccttcaaccaacatcttctgccctgggtcaagtgtacaaccgtcttcagatctcattgtatccagccttcagtcatcagacatgttcgttcttggagcaacatcccgtttatcactgtacccctcagtcgtacacatggcaagtccttcgcccaccgcagtgagctgaagcatgccaagagaatcgtggtgaagctcggcagtgccgtggtgacccgaggggatgaatgtggcctggccctggggcgcttggcatctattgttgagcaggtatcagtgctgcagaatcagggcagagagatgatgctggtgaccagtggagccgtagcctttggcaaacaaacgttgcgccatgagatccttctgtctcagagcgtgcggcaggccctccactcggggcagaaccagctgaaagaaatggcaattccagtcttagaggcacgagcctgtgcagctgccggacagagtgggctgatggccttgtatgaggctatgtttacccagtacagcatctgtgctgcccagattttggtgaccaatttggatttccatgatgagcagaagcgccggaacctcaatggaacacttcatgaactccttagaatgaacattgtccccattgtcaacacaaatgatgctgttgtccccccagctgagcccaacagtgacctgcagggggtaaatgttattagtgttaaagataatgatagcctggctgcccgactggctgtggaaatgaaaactgatctcttgattgtccttccagatgtagaaggcctttttgacagccccccaggttcagatgatgcaaagcttattgatatattttatcccggagatcagcagtctgtgacatttggacccaagtctagagtgggaaatgggtgcatggaagccaaggtgaaaagcaccctctgggctttgcaaggtggcacttctgttgttattgccaatggaacccacccaaaggtgtctgggcacgtcatcacagacattgtggaggggaagaaagttggtaccttcttttcagaagtaaagcctgcaggccctactgttgagcagcagggagaaatggcgcgatctggaggaaggatgttggccaccttggaacctgagcagagagcagaaattatccatcatctggctgatctgttgacggaccagcgtgatgagatcctgttagccaacaaaaaagacttggaggaggcagaggggagacttgcagctcctctgctgaaacgtttaagcctctccacatccaaattgaacagcctggccatcggtctgcgacagatcgcagcctcctcccaggacagcgtgggacgtgttttgcgccgcacccgaatcgccaaaaacttggaactggaacaagtgactgtcccaattggagttctgctggtgatctttgaatctcgtcctgactgtcctaccccaggtggcagctttgctatcgcaagtggcaatggcttgttactcaaaggagggaaggaggctgcacacagcaaccggattctccacctcctgacccaggaggctctctcaatccatggagtcaaggaggccgtgcaactggtgaataccagagaagaagttgaagatctttgccgcctagacaaaatgatagatctgatcattccacgtggctcttcccagctggtcagagacatccagaaagctgctaaggggattccagtgatggggcacagcgaagggatctgtcacatgtatgtggattccgaggccagtgttgataaggtcaccaggctagtcagagactctaaatgtgaatatccagctgcctgtaatgctttggagactttgttaatccaccgggatctgctcaggacaccattatttgaccagatcattgatatgctgagagtggaacaggtaaaaattcatgcaggccccaaatttgcctcctatctgaccttcagcccctccgaagtgaagtcactccgaactgagtatggggacctggaattatgcattgaagtagtggacaacgttcaggatgccattgaccacatccacaagtatggcagctcccacacggatgtcatcgtcacagaggacgaaaacacagcggagttcttcctgcagcacgtagacagtgcctgtgtgttctggaatgccagcactcgcttttctgatggttaccgctttggactgggagctgaagtgggaatcagtacatcgagaatccacgcccggggaccagtaggacttgagggactgcttactactaagtggctgctgcgagggaaggaccacgtggtctcagatttctcagagcatggaagtttaaaatatcttcatgagaacctccctattcctcagagaaacaccaactgaaaagagccaggaaaacccgggaattttccaaaaggtcttcacgttaaacttgtcttatctcaggagagagcccgctcttgtctcccagttcctggtagggtctgcctgttggaaagtgtacctggatgcttctgggctccgtttggcaatagcaatcttggctgatgtgcacagtctggctcccagctcaccctttttttttaaagtaagaaaatagttgctaccgatagggactttgccaagtccaattatcttctaggattgaaaggtgcattttccccataaaaaaggcgaggaaaacccatggctgctttgtgtcacctcagtgacttacagtcccccttggcatttagttggtactagagccagtatccttaacaa

SEQ ID NO: 34 - DNA, 3419 bp, Homo sapiens
  misc_feature (1)..(3419): downregulated in mutant
cccgggctcctcctctgccaaccaaccaggttgctaaaaatactacaacagcaaaagctgaaatatttgcagagaaaaactgaatttctgctacctccaccccctccactcaaatgggtaaaatctgacctaagaagccccccagatcacgctccaccaaagacacagggccttcaatactttctagcccccttctgattatgggtcctccacgggcttgagcaggatgtgaagttcagaaggtgccactcgagaaggggaagccacaagttattgggaaatagattgtcacgtgccttggcttttctgcctgcttactaggaccaaccctctccaaatccccttccatctgtcccaagaaaatctgtattggaccacaggagcttcccactaatccatcttttatcctctcttttgtctatccctcacctgccttggcttctattcctcacccctgtcatctccatgcttcctgctggctccatcccacggcacccaccaacatgccccagttgcctccctaacctggccattcccttccagagctcatctcctttccaggccagcagttttccttcatactatgggggctggagactgtaatgaaaaactttaaaatttgaaaagtcaagaacaactacgattccaacccatctggacagtaggtcatactccaaatgttgacggtggacccagcctaagccagcaggttacatgggatatacttgatcattctgccaagacaaactgcagagcagaccaccatccttaatgcatctgtctttgtcctagtactattgagtatcacaggtgagtcccaccagcccaccttgctgacaaaccccagccacttagaggtcagaagagggacctttccccagcgctagtgggtgccttggccccacagtggttaaggggcccacgctgagcatgttttggatgtcagtgggtgacaacccgtgcagggtgtgtgtctctgggtgtgtgggcagtgtgtgtgtctgcatctttgggtctgtgtgtgtaatttctatgaggccatatgtgttactgctcctgccactggtatttgagtttgtgagtgtctgtctttcatctcaggctctgccttcctctctgtcgtaggcgcttttcctcacctcagagcctgcccatcccccagggccagcactgtggccaagtgtttgcgtggctgttgctgaatggttgctttgcggatggaactgacagttgccgtatctgttctcctatggccctccagtgttcagagacaccctcaccccacacaaaggtgtcgttcagaagagggaagggctgtgctgcctgtgtgggagttggtggggggggtgttgtaaagatcccccttcagttctgaccagcaggaagaagggcccacaggcagcgcagtctgcagagggatgttgcctagcaacttgggagacttgactgcaaactcaccaaatcccgaattcctcagctctaatttgggaaaaaagcacagagtctaggcctggtatccactctgtcccccaatttccccctttcctctcctctctggattctggagttgaagtccagcaggaattggcacaacagaaggccacagatgtgcgtgtcatttaggcaagggcctgagttcccaggacatctggccacttggaggaggtggggagatgaaaatgtgccaaacagattggggaacagattttttgttacaaaagtgggtgggtccccaaacatgcgggagagaaaccctaacaccccaagccctggaatcccggccagcatccagttcagcctgggaccccaagtggctaaccttatcccaccctgaatggactcaggcatttctctcccactcatttttcctttggcaagcagagcctcgggcccaaacatttaggtggaaaggaagcatgtcaccgccacgtggtccctggtcatggccagggactggggatgcacagcagagctgtttcagggatatgctgtcccaaccagagccacagggcatttcccacctttgctcccagcaggagccatcctttcctccttcccttctctgggaccatagctgagaagacttttagttttttcagagaaggcctaccctacccacataacaagccacactgccagcttttgtaccctgccgagttgggggaggctgacgaccacaacagtggcagtctgtccctcccagatgctctttccatagctccccttcccagcctctcacgaacccacatcctgcctcatcatggacccaagagccaattacttcgaattctctacacttgggtttagggtattttttcttcttctcaggagcaagggcgaggatggccaagctttttgtcttattctgaggcagcagatcaaaaggttaggcagagtctggtatgaaagggaggaggatacttcttccaagtctgtgcccatatataaatacatacaaaataggtgttgtgcatatattgatgtgtttgttgcagctcatgtagacagatcgataatagatacgtcatgccgacagcttgagtttgggtgactgtccgtgtgcgtgcgtgaggcgggcgcccggccaaacagggtgtccgcatttacctgcaactgaagggctgcgtagttatttttgcaggttgtatctgcagtgtctgtatgctggaagaaggcctgggaaaatgatatgtgtggacatgtgggggcttgtgagcttttgtgtggacagcatgcccatccgccaggttgcaggagtgatgagacctttttaccgcccgttaaccaacaggttctccgtctgtagcaggagtgatgatgagacctttctttgcggactgagggggagtgaatgaaagggagccatatttgggactcctgctatggttgtggaagtatggagggatgggtgagggtgcgtggttttctctgagcgggagtaggtgctgtgcccagatgtgggtgtttgtgctcgggggtgtcgggggcctccttctgcctactccggcgtacatctgcctggcagtatgccgcctagcaagccaggggatttgcccgggagatggatggagtgtgtaggaggcttggatacaagcgtgtaggcggaggggctggggcaggagcgggacgcggcgcagagagcaccggagggcgtggcctccctccacttctccccgcccaccctgccggaggctgctcctgattggcttcccggccgcggggctcaggttacattcgcgagcggagccgagcgcgggagaccggacccgagagcagagctgctgtttcggcgcgggtcggctggcggccgatgccccagagcccccacccggcaccacacagaccccatccccgccctgcgccaccttcgtccccgcagaggacccccgacaccagcatggactgctgcaccgtaagtt

SEQ ID NO: 35 - DNA, 1987 bp, Homo sapiens
  misc_feature (1)..(1987): downregulated in mutant
acctgcagacccctaactcggtgctggtgatgggcctctctcccgcacccaatcctgcctgcacttctctttcctgaattgagacatgactgtcactccggcccctgaaccttcttcgccctgtcgctgcccagcccccagtgagtgtccctgccatagccctgctccaggctcgacaccccctctctgtacctcccaccccccgcgtcgccatagtccctgtccctcagggctctattcctaccccactcccgcggatcaatagccagtgaccccacaagaaccccccctgtcgcccccaggagaacgcctgctccaagccggacgacgacattctagacatcccgctggacgatcccggcgccaacgcggccgccgccaaaatccaggcgagttttcggggccacatggcgcggaagaagataaagagcggagagcgcggccggaagggcccgggccctggggggcctggcggagctggggtggcccggggaggcgcgggcggcggccccagcggagactaggccaggtgaggcgggcggcgcgcggctggctgacagctgcccttccacccagccctccccagagcaggggagaataaggcgggttggaggtgcagggggcgtggagggagctaagggttggggatagaaatccgagatgggaggtgggtgggaagaggctgaaggttgcgcggattccaggagctcacctgtttctccctctgcatcctcccttctgccccgccctggaccgaagaagaactgagcattttcaaaggtaatgaacacggccccctaggcggcgggggtggggaccgtgagtgggagaaaaggatcgaatcccagcccttccctctgcccaccctcaccctccagccggggaaatgcaccctgcccctgcgggttttgatacatccagacgcccgagctgcgacgggacatactgaccaagacatgcgcagccaccccatttggttgcacaagcccggccaaggggcagaaatacattgcggcgcgcacacgtacctgccgcggctcatttcagcgcttggtactcggggtgggggttctgggtgactgggccctctggcactcaccgcccaaagaatctggtactgaaagggggaagggtgggggaagaggagtagcagaagagggcaggttctcagaccttttctctcctcagttcccgaggagagatggatgccgcgtccccttcgcagcgacgagacttccctgccgtgtttgtgaccccctcctgcccagcaacctgccagctacaggagccccctgcgtcccagagactccctcacccaggcaggctccgtcgcggagtcgctgagtccgtgcccttttagttagttctgcagtctagtatggtccccatttgcccttccactccaccccaccctaaaccatgcgctcccaatcttccttcttttgcttctcgcccacctcttcccgcacccagcatgcagctctgcctccgcagcctcagtgcgctttcctgcgcgcactgcggagggcgccctaagcgtcacccaagcacactcacttaaagaaaaaacgagttctttcgttctgtgcgcagtaaaaggggcgccctacatctccgtgccactcccgccccagcctagccccaagactttggatccggggcgagatgaagggaagagggttgttttggtttcggacgacccttgctctgaccggaagagaagtccctatcccacacctgcctgtcacgttccctcccctttccccagcgcactgttgagggcagcctctccagctctcttgtttatgcaaacgccgagcgcctgggaggctcggtaggaggagtcttccacggccccgccccgccctgtcggtcccgccctccccccccgccgggctcctggggctgtggccgaaaggtttctgatctccgtgtgtgcatgtgactgtgctgggttggaatgtgaacaataaagaggaatgtccaa

SEQ ID NO: 36 - DNA, 176 bp, Homo sapiens
  misc_feature (1)..(176): downregulated in mutant
ctaggaaggatccaaggcagattttctttggtcttgctccaggatgggtggtgaatatggccgataagaaaatcctaaaacctacagatgaaaatctccttaagtattatacctcatgaattcccgtccaaggaagcagagttgttaaagagtactggaataggggctgaaggatc

SEQ ID NO: 37 - DNA, 3769 bp, Homo sapiens
  misc_feature (1)..(3769): downregulated in mutant
aaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaatttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtgatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactgctcctggtaacagtaatacattccattgttttagtaaccagaaatcttcatgcaatgaaaaatactttaattcatgaagcttactttttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 38 - DNA, 2564 bp, Homo sapiens
  misc_feature (1)..(2564): downregulated in mutant
gatttcagttgaaagatgtgtttttgtgagtagagcaccgcagaagaactgaagactgttgtgtgctccccgcagaaggggctaccatgatcctttcctcctataacaccatccagtcggttttctgttgctgctgttgctgttcagtgcagaagcgacaaatgagaacacagataagcctgagcacagatgaagagcttccagaaaaatacacccagcatcgcaggccgtggctcagccaattgtcaaataagaagcaatccaacacgggccgtgtgcagccgtcaaaacgaaagccactgcctcccctcccaccctctgaggttgctgaagagaagatccaagtcaaggcactttatgattttctgcccagagaaccctgtaatttagccttaaggagagcagaagaatacctgatactggagaaatacaatcctcactggtggaaggcaagagaccgtttggggaatgaaggcttaatcccaagcaactatgtgactgaaaacaaaataactaatttagaaatatatgagtggtaccatagaaacattaccagaaatcaggcagaacatctattgagacaagagtctaaagaaggtgcatttattgtcagagattcaagacatttaggatcctacacaatttccgtatttatgggagctagaagaagtacggaggctgccataaaacattatcagataaaaaagaatgactcaggacagtggtatgtggctgaaagacacgcctttcaatcaatccctgagttaatctggtatcaccagcacaatgcagccggtctcatgactcgtctccgatatccagttgggctgatgggcagttgtttaccagccacagctgggtttagctacgaaaagtgggagatagatccatctgagttggcttttataaaggagattggaagcggtcagtttggagtggtccatttaggtgaatggcggtcacatatccaggtagctatcaaggccatcaatgaaggctccatgtctgaagaggatttcattgaagaggccaaagtgatgatgaaattatctcattcaaagctagtgcaactttatggagtctgtatacagcggaagcccctttacattgtgacagagttcatggaaaatggctgcctgcttaactatctcagggagaataaaggaaagcttaggaaggaaatgctactgagtgtatgccaggatatatgtgaaggaatggaatatctggagaggaatggctatattcatagggatttggcggcaaggaattgtttggtcagttcaacatgcatagtaaaaatttcagactttggaatgacaaggtacgttttggatgatgagtatgtcagttcttttggagccaagttcccaatcaagtggtcccctcctgaagtttttcttttcaataagtacagcagtaaatctgatgtctggtcatttggagttttaatgtgggaagtttttacagaaggaaaaatgccttttgaaaataagtcaaatttgcaagtcgtggaagctatttctgaaggcttcaggctatatcgccctcacctggcaccaatgtccatatatgaagtcatgtacagctgctggcatgagaaacctgaaggccgccctacatttgcggagctgctgcgggctgtcacagagattgcggaaacctggtgaccggaaacagaatgccaacccaaagagtcatcttgcaaaactgtcatttattgtgaatatcttcaccatatggggtcacttatggtgaatatctttcttcagagttgctgactcttgaaaacagtgcaaagatcacagtttttaaaagttttaaaaatttaagaatattcacacaatcgtttttctatgtgtgagagggatttgcacactcttatttttctgtaaaatatttcacatcccaaatgtgaagaagtgaaaaagacttcgcagcagtcttcattgtggtgctcttcatgatcatagccccaggaacccttgaggttcttcttcacaaggctgagagtgcttccttcttgaagacgagtgtcattcatcacttcagtgatccatgcatagaatatgaaaataaattcttccaactcatgggataaaggggactcccttgaagaatttcatgtttttgggctgtatagctctttacagaaaatgcacctttataaatcacatgaatgttagtattctggaaatgtcttttgttaatataatcttcccatgttatttaacaaattgtttttgcacatatctgattatattgaaagcagtttttttgcattcgagttttaaacactgttataaaatgtagccaaagctcacctttgaacagatcccggtgacattctatttccaggaaaatccggaacctgattttagttctgtgattttacactttttacatgtgagattggacagtttcagaggccttattttgtcatactaagtgtctcctgtaattttcaggaagatgatttgttctttccagaagaggagacaaaagcaagatagccaaatgtgacatcaagctccattgtttcggaaatccaggattttgaattc